(19) United States
(12) Patent Application Publication
(10) Pub. No.: US 2025/0043275 A1
(43) Pub. Date: Feb. 6, 2025

(54) METHODS OF PREPARING LOOP FORK LIBRARIES

(71) Applicant: Illumina, Inc., San Diego, CA (US)

(72) Inventors: Eli M. Carrami, Cambridge (GB); Jonathan Mark Boutell, Bishops Stortford (GB); Oliver Jon Miller, Linton (GB); Aathavan Karunakaran, Berkeley, CA (US); Stephen Paul Bruinsma, Madison, WI (US); Niall Anthony Gormley, Cambridge (GB)

(21) Appl. No.: 18/885,481

(22) Filed: Sep. 13, 2024

Related U.S. Application Data
(63) Continuation of application No. PCT/EP2023/056641, filed on Mar. 15, 2023.
(60) Provisional application No. 63/439,519, filed on Jan. 17, 2023.
(60) Provisional application No. 63/439,415, filed on Jan. 17, 2023.
(60) Provisional application No. 63/439,438, filed on Jan. 17, 2023.
(60) Provisional application No. 63/439,466, filed on Jan. 17, 2023.
(60) Provisional application No. 63/439,501, filed on Jan. 17, 2023.
(60) Provisional application No. 63/439,417, filed on Jan. 17, 2023.
(60) Provisional application No. 63/439,443, filed on Jan. 17, 2023.
(60) Provisional application No. 63/439,491, filed on Jan. 17, 2023.
(60) Provisional application No. 63/439,522, filed on Jan. 17, 2023.
(60) Provisional application No. 63/269,383, filed on Mar. 15, 2022.

(51) Int. Cl. C12N 15/10

(52) U.S. Cl. CPC C12N 15/1068; C12N 15/1065

(57) ABSTRACT

The invention relates to methods and kits for use in nucleic acid sequencing, in particular methods for use in concurrent sequencing, and in particular concurrent sequencing of tandem insert libraries.

### RELATED APPLICATIONS

This application is a continuation application of PCT Application No. PCT/EP2023/056641 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023; the contents of which are incorporated by reference in their entireties.

### REFERENCE TO SEQUENCE LISTING

The present application is being filed along with a Sequence Listing in electronic format. The Sequence Listing is provided as a file entitled IP_2569_SEQLLIST.xml, created Sep. 13, 2024, which is approximately 77,824 bytes in size. The information in the electronic format of the Sequence Listing is incorporated herein by reference in its entirety.

### FIELD OF THE INVENTION

The invention relates to methods and kits for use in nucleic acid sequencing, in particular methods for use in concurrent sequencing, and in particular concurrent sequencing of tandem insert libraries. Further, the invention relates to methods of detecting mismatched base pairs in nucleic acid sequences

### BACKGROUND

The common expectation is that the complementary sequences of a double-stranded DNA molecule should carry identical information, and as such, sequencing one strand of the molecule should be sufficient to determine the sequence. In practice, however, this notion is not accurate. The most common occasion where the symmetry of information between complementary strands may break is due to DNA damage. Different bases of DNA have different susceptibilities to different forms of damage. For instance, G is very sensitive to oxidative damage leading to the formation of oxo-G, the formation of which is one of the main reasons of library prep dependent sequencing errors, as DNA polymerases often unfaithfully pair oxo-G with A, leading to high quality C>A sequencing errors. Another situation in which the symmetry of information between the strands may break is during methyl-C (mC) sequencing. Standard protocols modify C or mC to alternative bases such as U, thereby changing the sequence information only in one strand.

Various strategies have been proposed to enable the sequencing of both strands of a double-stranded DNA molecule, commonly known as duplex sequencing.

Original methods of duplex sequencing used bioinformatics methods or high-depth sequencing data to identify clusters corresponding to each of the strands in original template DNA molecules and used this information to correct potential sequencing errors. Other methods used physical separation or UMI index sequences to discriminately label strands of DNA that originate from the same double-stranded template. Naturally, such methods are either very complex or are inefficient at identifying the correct duplex molecules.

Recently, a more efficient strategy for generating duplex sequencing information for the purpose of sequencing error correction was proposed. This method generates tandem insert libraries containing the sequence information from each strand of a double-stranded template in a direct repeat fashion. The direct repeat format of this library is essential for its functionality as it avoids the rehybridization of the sequencing template during sequencing by synthesis (SBS). This method, while compatible with SBS, suffers from very low conversion efficiency during library preparation.

There therefore exists a need to develop improved methods that can sequence both strands of a double-stranded DNA molecule (duplex sequencing), and in particular a need for methods that are compatible with SBS.

### SUMMARY OF THE INVENTION

According to an aspect of the present invention, there is provided a method of preparing at least one polynucleotide library strand template, wherein the method comprises:
attaching a first adaptor to a first end of a double-stranded polynucleotide sequence, wherein the first end comprises the 3′ end of the forward strand and the 5′ end of the reverse strand of the double-stranded polynucleotide sequence; and
attaching a second adaptor to a second end of a double-stranded polynucleotide sequence, wherein the second end comprises the 5′ end of the forward strand and the 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease and/or the second adaptor further comprises at least one cleavable site and/or a complement of a cleavable site.

In one embodiment, the first adaptor comprises a base-paired stem and a loop, wherein the first restriction site is in the base-paired stem. Alternatively or additionally, the first restriction site is in the loop.

In one embodiment, the first restriction site is a restriction site for a nicking endonuclease or a restriction endonuclease.

In one embodiment, the second adaptor further comprises at least one cleavable site and/or a complement of a cleavable site. In one example, the second adaptor comprises a base-paired stem and a fork, wherein the fork comprises a primer-binding complement sequence and a primer-binding sequence. In one embodiment, the cleavable site and/or a complement of a cleavable site is in the base-paired stem. In an alternative embodiment, the second adaptor comprises a base-paired stem and a loop, wherein the loop comprises a second cleavable site.

In one embodiment, the at least one cleavable site and/or a complement of a cleavable site is a restriction site for a nicking endonuclease, wherein the restriction site may be a second restriction site.

In one embodiment, the first adaptor further comprises an affinity tag.

In another aspect of the invention there is provided a polynucleotide library strand for sequencing comprising a first adaptor, a double-stranded polynucleotide sequence to be identified and a second adaptor;
wherein the first adaptor is attached to a first end of the double-stranded polynucleotide sequence, wherein the first end comprises the 3′ end of the forward strand and the 5′ end of the reverse strand of the double-stranded polynucleotide sequence; and
wherein the second adaptor is attached to a second end of the double-stranded polynucleotide sequence, wherein the second end comprises the 5′ end of the forward strand and the 3′ end of the reverse strand of the double-stranded polynucleotide sequence;
wherein the first adaptor comprises a base-paired stem and a loop; and
wherein the second adaptor comprises a base-paired stem, a primer-binding complement sequence and a primer-binding sequence; and
wherein the first adaptor comprises at least one restriction site for an endonuclease.

In one embodiment, the second adaptor comprises at least one cleavable site and/or a complement of a cleavable site, wherein the cleavable site and/or a complement of a cleavable site may be a restriction site for a nicking endonuclease.

In another aspect of the invention, there is provided a method of identifying at least a first region of a polynucleotide sequence, wherein the method comprises:
a. preparing at least one polynucleotide library strand as described above;
b. amplifying the polynucleotide library strand to generate a first and second library strand, wherein each library strand comprises a first and second region;
c. hybridising the first or second library strands to first and second immobilised primers respectively on a solid support and carrying out a first extension reaction to generate a first or second immobilised template strand;
d. hybridising the first or second immobilised template strands to a second or first immobilised primer respectively and carrying out a second extension reaction to generate a second and first immobilised template strand;
e. hybridising the first and second immobilised template strands;
f. applying a first endonuclease; and
g. sequencing the first and second immobilised template strands, wherein sequencing the first and second immobilised template strands identifies the first region.

In one embodiment, identifying comprises determining the sequences of a first region and/or identifying any epigenetic modification, wherein the epigenetic modification may be a modified cytosine.

In one embodiment, each first and second library strands comprise a primer-binding complement sequence, a first portion, a first adaptor sequence, a second portion and a primer-binding sequence, and wherein the first adaptor comprises a first restriction site for an endonuclease.

In one embodiment, the first restriction site is a restriction site for a nicking endonuclease or a restriction endonuclease.

In one embodiment, the primer-binding sequence and primer-binding complement sequence comprise at least one cleavable and/or a complement of a cleavable site. In one embodiment, the cleavable site and/or a complement of a cleavable site is a second restriction site.

In one embodiment, following cleavage of the first restriction site, non-immobilised library strands are de-hybridised and the immobilised template strands are sequenced by single-stranded SBS (sequencing by synthesis). Alternatively, following cleavage of the first restriction site, the immobilised template strands are sequenced by double-stranded SBS (sequencing by synthesis).

In one embodiment, the at least one nicking endonuclease cleaves the second restriction site and the immobilised strands are sequenced by double-stranded SBS (sequencing by synthesis).

In one embodiment, the method further comprises blocking all or substantially all 3′ ends of the sequenced immobilised strands.

In one embodiment, the method further comprises applying a second nicking endonuclease and sequencing the first and second immobilised template strands identifies the second region, wherein the second nicking endonuclease cleaves a different restriction site from the first nicking endonuclease.

In one embodiment, the method further comprises carrying out an extension reaction to regenerate the first and second immobilised strands.

In one embodiment, the method further comprises applying a second nicking endonuclease and sequencing the first and second immobilised template strands identifies the second region, wherein the second nicking endonuclease cleaves a different restriction site from the first nicking endonuclease.

In another aspect of the invention there is provided an inverted-repeat tandem-insert polynucleotide library strand for sequencing, wherein the library strand comprises a primer-binding complement sequence, a first portion to be identified, a first adaptor sequence, a second portion to be identified and a primer-binding sequence, wherein the sequence of the second portion is inverted with respect to the first portion, and wherein the loop sequence comprises at least one restriction site.

In another aspect of the invention there is provided a library preparation kit comprising of a plurality of first adaptors and a plurality of second adaptors, wherein the first adaptors comprise a base-paired stem and a loop, and wherein the first adaptors comprise at least one restriction site, and wherein the second adaptors comprise a base-paired stem, a primer-binding sequence and a primer-binding complement sequence, wherein optionally the second adaptors comprise at least one restriction site.

### DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a typical solid support.

FIG. 2 shows the stages of bridge amplification and the generation of an amplified cluster comprising (A) a library strand hybridising to an immobilised primer; (B) generation of a template strand from the library strand; (C) dehybridisation and washing away the library strand; (D) hybridisation of the template strand to another immobilised primer; (E) generation of a template complement strand from the template strand via bridge amplification; (F) dehybridisation of the sequence bridge; (G) hybridisation of the template strand and template complement strand to immobilised primers; and (H) subsequent bridge amplification to provide a plurality of template and template complement strands.

FIG. 3 shows the detection of nucleobases using 4-channel, 2-channel and 1-channel chemistry.

FIG. 4 shows starting from a double-stranded polynucleotide sequence comprising a forward strand of the sequence and a reverse strand of the sequence adaptors may be ligated to generate a loop fork ligated polynucleotide sequence and subsequent amplification using PCR to generate a self-tandem insert library.

FIG. 5 shows three adaptor configurations are produced after ligation of the adapters, one which represents the desired loop/fork configuration. PCR and/or clustering steps eliminate the loop/loop configuration, due to it lacking any primer binding sites. A single affinity-based system eliminates unwanted fork/fork molecules.

FIG. 6 shows the binding of primers to a primer-binding sequence on a template duplex, thus preparing a tandem library fragment for sequencing.

FIG. 7 shows that a 9QAM encoding scheme can be used to accurately differentiate between two simultaneously received base calls; plotting relative intensities of light signals obtained from Read 1.1 and Read 1.2 generates a constellation of 9 clouds. The four corner clouds represent high quality and accurate base calls, while off-corner clouds represent potential library prep/sequencing errors, which could be eliminated.

FIG. 8 shows that a 9QAM encoding scheme can be used to simultaneously sequence genomic and epigenetic data; epigenetic conversion of the polynucleotide library strand by, for example, Bisulfite/EM-Seq or TAPS and subsequent sequencing enables mC and the canonical bases to be identified simultaneously.

FIG. 9 shows an exemplar nicking arrangement to facilitate sequencing of the entire inverted-repeat tandem-insert duplex. Following nicking of the lawn primers and sequencing of the first strand (read 1), the free ends of the sequenced strands are blocked. Nicking enzymes, specific for an alternative recognition site, are added to nick a recognition site within the loop sequence to generate two start sites for simultaneous sequencing of the other strand of the original polynucleotide duplex.

FIG. 10 shows an exemplar nicking arrangement to facilitate sequencing of the entire inverted-repeat tandem-insert duplex. The first nicking event can occur within the loop sequence, and the polynucleotides sequences dehybridized for the first read. The sequenced strands are extended to regenerate the 3′ primer-binding sequences. Nicking enzymes may be applied to nick the lawn primers, to produce two sequencing start sites that allow simultaneous sequencing from opposite end of both inserts

FIG. 11 shows a nick arrangement at the loop sequence that generates two immobilised extended strands, effectively halving the tandem insert. Following dehybridisation, a first and second sequencing primers can be applied and bind to their respective primer-binding sequences to facilitate Read 1.1 and Read 1.2.

FIG. 12 shows an example of a method of sequencing an inverted-repeat tandem-insert library strand. Following library preparation, cluster generation occurs and a loop-hybridised sequence bridge forms. Nicking enzymes may be applied to nick the sequence bridges at a pair of recognition sequences in the loop stem simultaneously, providing sequencing start sites for different strands of the original duplex template. The strands can be simultaneously sequenced by standard SBS or double-stranded SBS (e.g. strand displacement SBS). In standard SBS sequencing, the non-immobilised sequences—i.e., the sequences 3′ of the nicked site—are washed off before the sequences steps for R1.1 and R1.2. In double-stranded SBS (e.g. strand displacement SBS), the non-immobilised sequences 3′ of the nicked site are not washed off.

FIG. 13 is a plot showing graphical representations of sixteen distributions of signals generated by polynucleotide sequences according to one embodiment.

FIG. 14 is a flow diagram showing a method for base calling according to one embodiment.

FIG. 15 is a plot showing graphical representations of nine distributions of signals generated by polynucleotide sequences according to one embodiment.

FIG. 16 shows the effect of unmodified cytosine to uracil conversion treatment of a double-stranded polynucleotide, and a scatter plot showing the resulting distributions of signals generated by polynucleotide sequences.

FIG. 17 shows the effect of modified cytosine to thymine conversion treatment of a double-stranded polynucleotide, and a scatter plot showing the resulting distributions of signals generated by polynucleotide sequences.

FIG. 18 shows alternative signal distributions using a different dye-encoding scheme.

FIG. 19 shows alternative signal distributions using a different dye-encoding scheme.

FIG. 20 shows alternative signal distributions using a different dye-encoding scheme.

FIG. 21 is a flow diagram showing a method for determining sequence information according to one embodiment.

FIG. 22 shows 9 QaM analysis conducted on the signals obtained from the custom second hyb run of Example 1. The x-axis shows signal intensity from a “red” wavelength channel, whilst the y-axis shows signal intensity from a “green” wavelength channel. G is not associated with any dyes and as such appears contributes no intensity for both “red” and “green” channels. C is associated with a “red” dye and as such contributes intensity to the “red” channel, but not the “green” channel. T is associated with a “green” dye and as such contributes intensity to the “green” channel, but not the “red channel. A is associated with both a “red” dye and a “green” dye, and as such contributes intensity to both the “red” channel and “green” channel. Since the template comprises forward and reverse complement strands that are sequenced simultaneously, most of the readout will generate (G,G) read (bottom left corner), (C,C) read (bottom right corner), (T,T) read (top left corner), and (A,A) read (top right corner) clouds. However, a central cloud corresponding to (C,T) or (T,C) reads corresponds with the presence of modified cytosines. FIG. 22B shows sequence data generated from two different primers used (HYB2′-ME and HP10) in the custom second hyb run of Example 1. Mismatches between the two sequences allow identification of modified cytosines. For example, 5-mC present in the original forward strand of the target polynucleotide is read as T in the HP10 read, whereas C present in the original reverse complement strand of the target polynucleotide (corresponding to the same position as 5-mC in the original forward strand of the target polynucleotide) is read as C in the HYB2′-ME read.

FIGS. 23A to 23F show 9 QaM analysis conducted on the signals obtained from Example 2 (library fragments 1 to 6). The x-axis shows signal intensity from a “red” wavelength channel, whilst the y-axis shows signal intensity from a “green” wavelength channel. A CA dye swap has been performed in this MiniSeq run compared to a standard MiniSeq run. G is not associated with any dyes and as such appears contributes no intensity for both “red” and “green” channels. A is associated with a “red” dye and as such contributes intensity to the “red” channel, but not the “green” channel. T is associated with a “green” dye and as such contributes intensity to the “green” channel, but not the “red channel. C is associated with both a “red” dye and a “green” dye, and as such contributes intensity to both the “red” channel and “green” channel. Since the template comprises forward and reverse complement strands that are sequenced simultaneously, the readout will generate (T,T) reads (top left corner), (T,C) reads (top middle), (C,C) reads (top right corner), (G,G) reads (bottom left corner), (G,A) reads (bottom middle), and (A,A) reads (bottom right corner). The top right corner corresponds to a (5-mC)-G base pair, whilst the bottom left corner corresponds to a G-(5-mC) base pair, thus corresponding with the presence of modified cytosines. Groupings are as follows: T in forward strand of library in top left (marked as “T”); C in forward strand of library in top middle (marked as “C”); 5-mC in forward strand of library in top right (marked as “c”); G in forward strand of library and associated with 5-mC in reverse strand of library in bottom left (marked as “g”); G in forward strand of library and associated with C in reverse strand of library in bottom middle (marked as “G”); and A in forward strand of library in bottom right (marked as “A”). In FIGS. 23A to 23C, two scatter-plots are shown: the plot marked “read-color coded” corresponds to assignments for each base to particular groups during the read process; the plot marked “ref-color coded” shows the true assignments for each base to particular groups and is indicative of where errors have occurred in the read process. FIGS. 23D to 23F show combined “read-color coded” and “ref-color coded” plots—where the read and the reference differ, a border is shown for the read assignment, whilst the central portion of the circle shows the actual assignment. In addition, FIGS. 23A to 23F show sequence alignment of the read sequence to the true methylated pUC19 sample-“m” above or below a C represents 5-mC, whilst “m” above or below a G represents G that is base-paired with 5-mC; red boxes indicate errors in read (of sequence or methylation status).

### DETAILED DESCRIPTION OF THE INVENTION

All patents, patent applications, and other publications referred to herein, including all sequences disclosed within these references, are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

The present invention can be used in sequencing, in particular duplex sequencing. Methodologies applicable to the present invention have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US 20060024681, US20060292611, WO 06/110855, WO 06/135342, WO 03/074734, WO07/010252, WO 07/091077, WO 00/179553, WO 98/44152 and WO 2022/087150, the contents of which are herein incorporated by reference.

As used herein, the term “variant” refers to a variant polypeptide sequence or part of the polypeptide sequence that retains desired function of the full non-variant sequence. For example, a desired function of the immobilised primer retains the ability to bind (i.e. hybridise) to a target sequence.

As used in any aspect described herein, a “variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid sequence. The sequence identity of a variant can be determined using any number of sequence alignment programs known in the art. As an example, Emboss Stretcher from the EMBL-EBI may be used: https://www.ebi.ac.uk/Tools/psa/emboss_stretcher/(using default parameters: pair output format, Matrix=BLOSUM62, Gap open=1, Gap extend=1 for proteins; pair output format, Matrix=DNAfull, Gap open=16, Gap extend=4 for nucleotides). As used herein, the term “fragment” refers to a functionally active series of consecutive nucleic acids from a longer nucleic acid sequence. The fragment may be at least 99%, at least 95%, at least 90%, at least 80%, at least 70%, at least 60%, at least 50%, at least 40% or at least 30% the length of the longer nucleic acid sequence. A fragment as used herein may also retain the ability to bind (i.e. hybridise) to a target sequence.

Sequencing typically comprises four fundamental steps: 1) library preparation to form a plurality of target polynucleotides for identification; 2) cluster generation to form an array of amplified template polynucleotides; 3) sequencing the cluster array of amplified template polynucleotides; and 4) data analysis to identify characteristics of the target polynucleotides from the amplified template polynucleotide sequences. These steps are described in greater detail below.

### Library Strands and Template Terminology

For a given double-stranded polynucleotide sequence (also referred to herein as a polynucleotide library) to be identified, the polynucleotide sequence comprises a forward strand of the sequence and a reverse strand of the sequence.

Typically, when the polynucleotide sequence is replicated (e.g. using a DNA/RNA polymerase), complementary versions of the forward strand of the sequence and the reverse strand of the sequence are generated. These may be referred to as the forward complement strand of the sequence and the reverse complement strand of the sequence respectively.

By using the forward complement strand of the sequence as a template for complementary base pairing, a sequencing process (e.g. a sequencing-by-synthesis or a sequencing-by-ligation process) reproduces information that was present in the original forward strand of the sequence. The forward complement strand of the sequence may be referred to as the forward strand of the template.

Similarly, by using the reverse complement strand of the sequence as a template for complementary base pairing, a sequencing process (e.g. a sequencing-by-synthesis or a sequencing-by-ligation process) reproduces information that was present in the original reverse strand of the sequence. The reverse complement strand of the sequence may be referred to as the reverse strand of the template.

### Library Preparation

Library preparation is the first step in any high-throughput sequencing platform. These libraries allow templates to be generated via complementary base pairing that can subsequently be clustered and amplified. During library preparation, nucleic acid sequences, for example genomic DNA sample, or cDNA or RNA sample, are converted into polynucleotide templates, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). The original sample DNA fragments are referred to as “inserts”. The target polynucleotides may advantageously also be size-fractionated prior to modification with the adaptor sequences.

As described herein, typically the templates to be generated from the libraries are duplexes comprising a first portion, that is the forward strand (of the template) and a second portion, that is the reverse strand (of the template). Generating these templates from particular libraries may be performed according to methods known to persons of skill in the art. However, some example approaches of preparing libraries suitable for generation of such templates are described below.

In some embodiments, the library is prepared by ligating adaptor sequences to the duplex, as described in more detail in e.g. WO 07/052006, which is incorporated herein by reference. In some cases, “tagmentation” can be used to attach the sample DNA to the adaptors, as described in more detail in e.g. WO 10/048605, US 2012/0301925, US 2013/0143774 and WO 2016/189331, each of which are incorporated herein by reference. In tagmentation, double-stranded DNA is simultaneously fragmented and tagged with adaptor sequences and PCR primer binding sites. The combined reaction eliminates the need for a separate mechanical shearing step during library preparation.

Where below features are described in relation to the “forward” strand, it should be considered that these features could equally be applied to the “reverse strand”.

In one embodiment, as described in further detail below, the library may be prepared using a loop fork method, which is described below. This procedure may be used, for example, for preparing templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template). This procedure may also be used, for example, for preparing templates comprising concatenated polynucleotide sequences, wherein a single sequence comprises both the forward and reverse strands of the template—or a copy of the forward strand of the template (i.e. a forward complement strand of the template) and a copy of the reverse strand of the template (i.e. a reverse complement strand of the template). In one aspect, the present invention describes methods of preparing an inverted-repeat tandem-insert polynucleotide, where the orientation of the forward strand with respect to the reverse strand (or the copy of the forward strand with respect to the reverse strand) is inverted.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, or a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

As used herein, an “adaptor” refers to a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the P5′ and P7′ primer-binding sequences are complementary to short primer sequences (or lawn primers) present on the surface of a flow cell. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The loop complement (or the loop) may comprise an internal sequencing primer binding site. In other words, an internal sequencing primer binding site may form part of the loop complement. Alternatively, the loop complement may be an internal sequencing primer binding site. Accordingly, we may refer to the loop complement herein as comprising a second sequencing primer binding site, or as a second sequencing primer binding site.

### Cluster Generation and Amplification

Once a double stranded nucleic acid template is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example P5 and P7 lawn primers).

Thus, embodiments of the present invention may be performed on a solid support 200, such as a flowcell. However, in alternative embodiments, seeding and clustering can be conducted off-flowcell using other types of solid support.

The solid support 200 may comprise a substrate 204. See FIG. 1. The substrate 204 comprises at least one well 203 (e.g. a nanowell), and typically comprises a plurality of wells 203 (e.g. a plurality of nanowells).

In one embodiment, the solid support comprises at least one first immobilised primer and at least one second immobilised primer. These immobilised primers may also be known as lawn primers.

Thus, each well 203 may comprise at least one first immobilised primer 201, and typically may comprise a plurality of first immobilised primers 201. In addition, each well 203 may comprise at least one second immobilised primer 202, and typically may comprise a plurality of second immobilised primers 202. Thus, each well 203 may comprise at least one first immobilised primer 201 and at least one second immobilised primer 202, and typically may comprise a plurality of first immobilised primers 201 and a plurality of second immobilised primers 202.

The first immobilised primer 201 may be attached via a 5′-end of its polynucleotide chain to the solid support 200. When extension occurs from the first immobilised primer 201, the extension may be in a direction away from the solid support 200.

The second immobilised primer 202 may be attached via a 5′-end of its polynucleotide chain to the solid support 200. When extension occurs from second immobilised primer 202, the extension may be in a direction away from the solid support 200.

The first immobilised primer 201 may be different to the second immobilised primer 202 and/or a complement of the second immobilised primer 202. The second immobilised primer 202 may be different to the first immobilised primer 201 and/or a complement of the first immobilised primer 201.

The (or each of the) first immobilised primer(s) 201 may comprise a sequence as defined in SEQ ID NO. 1 or 5, or a variant or fragment thereof. The second immobilised primer(s) 202 may comprise a sequence as defined in SEQ ID NO. 2, or a variant or fragment thereof.

By way of brief example, following attachment of the P5 and P7 primers to the solid support, the solid support may be contacted with the template to be amplified under conditions which permit hybridisation (or annealing-such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. The resulting structure is referred to herein as a sequence bridge. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products (or sequence bridges). This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

In some cases, to facilitate sequencing, one set of strands (either the original template strands or the complement strands thereof) may be removed from the solid support leaving either the original template strands or the complement strands. Suitable methods for removing such strands are described in more detail in application number WO 07/010251, the contents of which are incorporated herein by reference in their entirety.

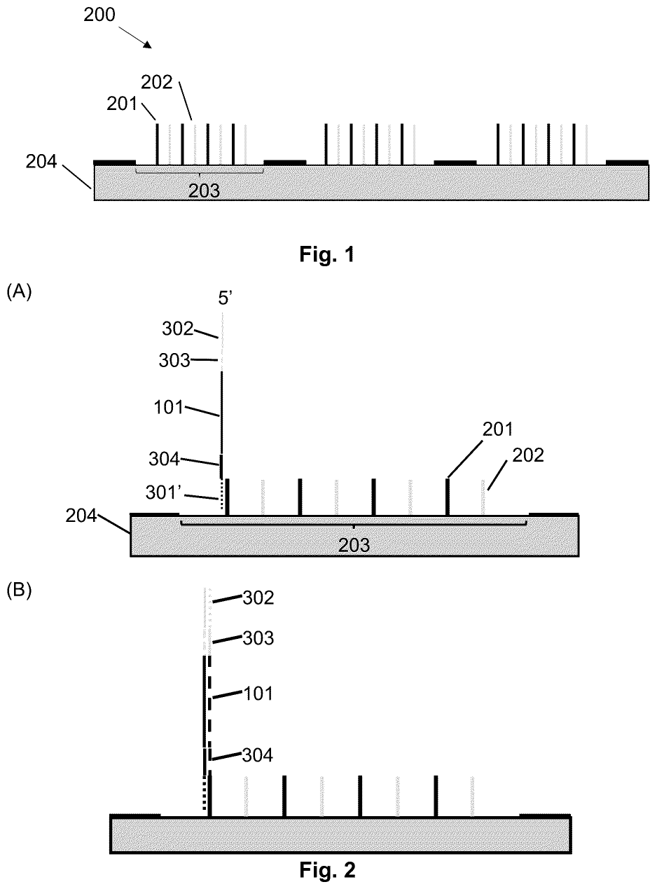

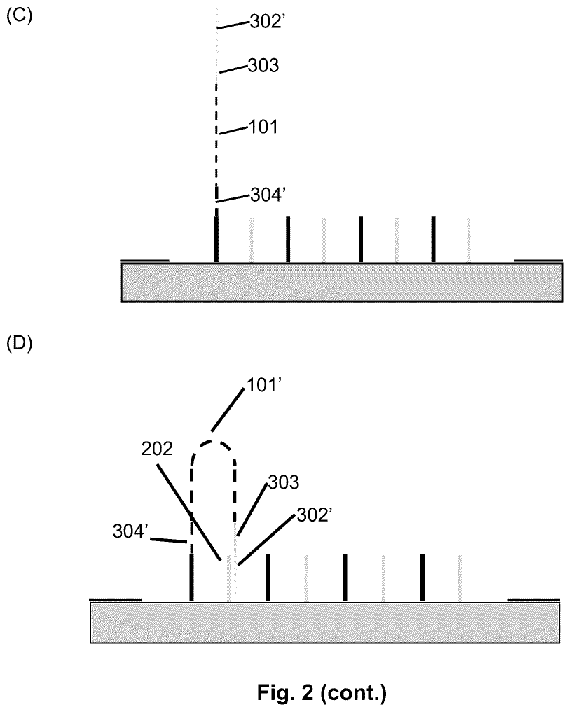

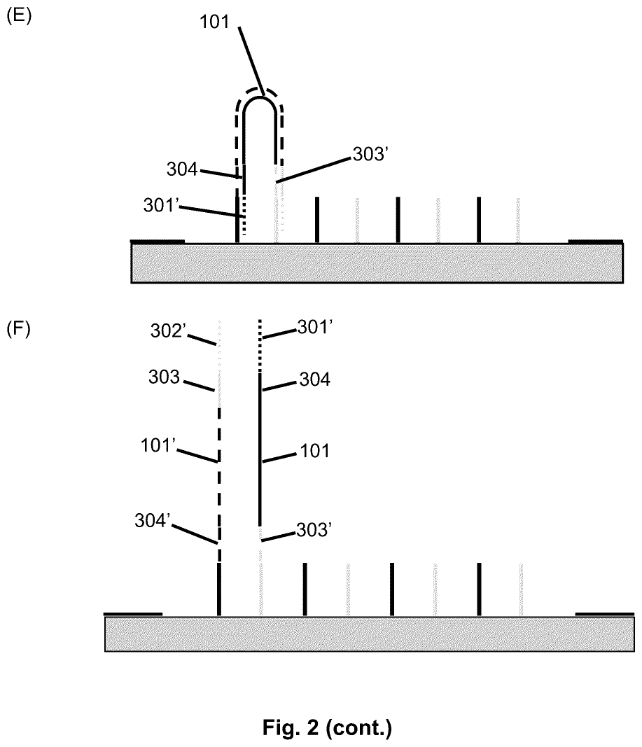

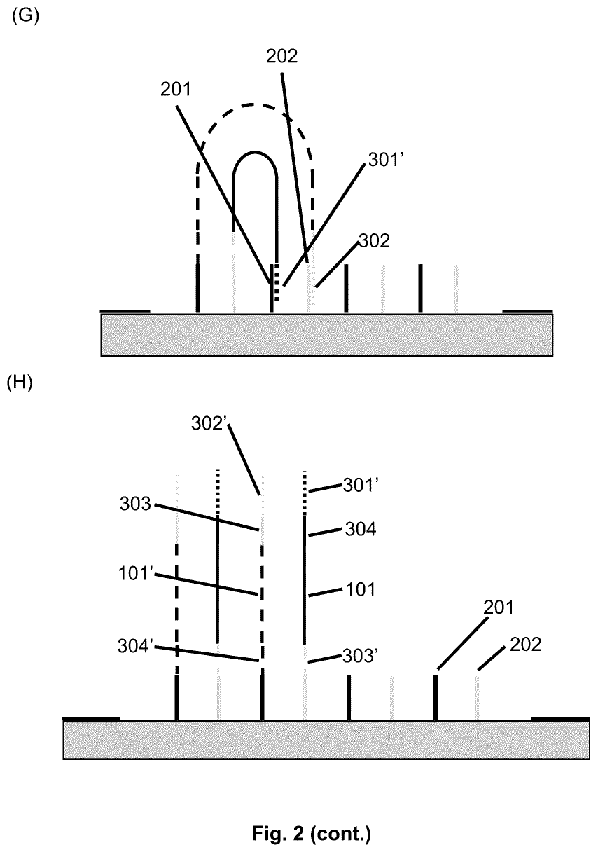

The steps of cluster generation and amplification for templates comprising a first portion and a second portion are illustrated below and in FIG. 2.

### Sequencing

As described herein, the template provides information (e.g. identification of the genetic sequence, identification of epigenetic modifications) on the original target polynucleotide sequence. For example, a sequencing process (e.g. a sequencing-by-synthesis (referred to herein as SBS) or sequencing-by-ligation process) may reproduce information that was present in the original target polynucleotide sequence, by using complementary base pairing.

In one embodiment, sequencing may be carried out using any suitable “sequencing-by-synthesis” technique, wherein nucleotides are added successively in cycles to the free 3′ hydroxyl group, resulting in synthesis of a polynucleotide chain in the 5′ to 3′ direction. The nature of the nucleotide added may be determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. Such a label may be configured to emit a signal, such as an electromagnetic signal, or a (visible) light signal.

In a particular embodiment, the label is a fluorescent label (e.g. a dye). Thus, such a label may be configured to emit an electromagnetic signal, or a (visible) light signal. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

However, the detectable label need not be a fluorescent label. Any label can be used which allows the detection of the incorporation of the nucleotide into the DNA sequence.

Each cycle may involve simultaneous delivery of four different nucleotide types to the array of template molecules. Alternatively, different nucleotide types can be added sequentially and an image of the array of template molecules can be obtained between each addition step.

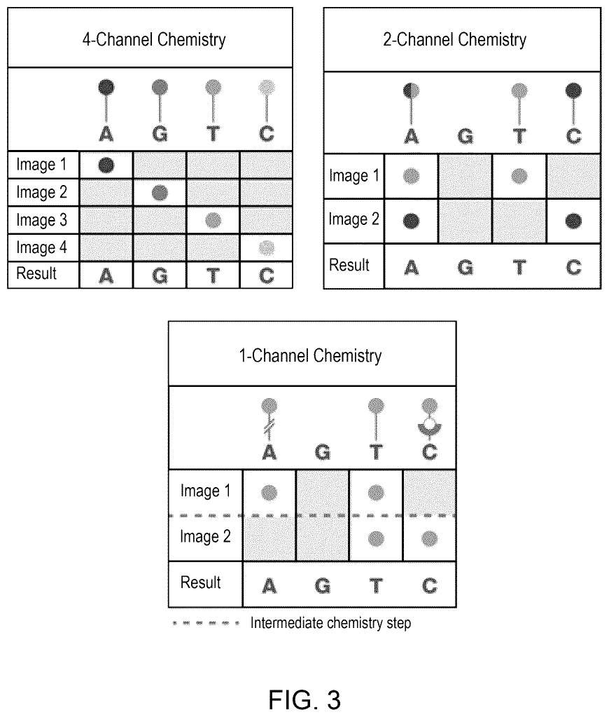

In some embodiments, each nucleotide type may have a (spectrally) distinct label. In other words, four channels may be used to detect four nucleobases (also known as 4-channel chemistry) (FIG. 3—left). For example, a first nucleotide type (e.g. A) may include a first label (e.g. configured to emit a first wavelength, such as red light), a second nucleotide type (e.g. G) may include a second label (e.g. configured to emit a second wavelength, such as blue light), a third nucleotide type (e.g. T) may include a third label (e.g. configured to emit a third wavelength, such as green light), and a fourth nucleotide type (e.g. C) may include a fourth label (e.g. configured to emit a fourth wavelength, such as yellow light). Four images can then be obtained, each using a detection channel that is selective for one of the four different labels. For example, the first nucleotide type (e.g. A) may be detected in a first channel (e.g. configured to detect the first wavelength, such as red light), the second nucleotide type (e.g. G) may be detected in a second channel (e.g. configured to detect the second wavelength, such as blue light), the third nucleotide type (e.g. T) may be detected in a third channel (e.g. configured to detect the third wavelength, such as green light), and the fourth nucleotide type (e.g. C) may be detected in a fourth channel (e.g. configured to detect the fourth wavelength, such as yellow light). Although specific pairings of bases to signal types (e.g. wavelengths) are described above, different signal types (e.g. wavelengths) and/or permutations may also be used.

In some embodiments, detection of each nucleotide type may be conducted using fewer than four different labels. For example, sequencing-by-synthesis may be performed using methods and systems described in US 2013/0079232, which is incorporated herein by reference.

Thus, in some embodiments, two channels may be used to detect four nucleobases (also known as 2-channel chemistry) (FIG. 3—middle). For example, a first nucleotide type (e.g. A) may include a first label (e.g. configured to emit a first wavelength, such as green light) and a second label (e.g. configured to emit a second wavelength, such as red light), a second nucleotide type (e.g. G) may not include the first label and may not include the second label, a third nucleotide type (e.g. T) may include the first label (e.g. configured to emit the first wavelength, such as green light) and may not include the second label, and a fourth nucleotide type (e.g. C) may not include the first label and may include the second label (e.g. configured to emit the second wavelength, such as red light). Two images can then be obtained, using detection channels for the first label and the second label. For example, the first nucleotide type (e.g. A) may be detected in both a first channel (e.g. configured to detect the first wavelength, such as red light) and a second channel (e.g. configured to detect the second wavelength, such as green light), the second nucleotide type (e.g. G) may not be detected in the first channel and may not be detected in the second channel, the third nucleotide type (e.g. T) may be detected in the first channel (e.g. configured to detect the first wavelength, such as red light) and may not be detected in the second channel, and the fourth nucleotide type (e.g. C) may not be detected in the first channel and may be detected in the second channel (e.g. configured to detect the second wavelength, such as green light). Although specific pairings of bases to signal types (e.g. wavelengths) and/or combinations of channels are described above, different signal types (e.g. wavelengths) and/or permutations may also be used.

In some embodiments, one channel may be used to detect four nucleobases (also known as 1-channel chemistry) (FIG. 3—right). For example, a first nucleotide type (e.g. A) may include a cleavable label (e.g. configured to emit a wavelength, such as green light), a second nucleotide type (e.g. G) may not include a label, a third nucleotide type (e.g. T) may include a non-cleavable label (e.g. configured to emit the wavelength, such as green light), and a fourth nucleotide type (e.g. C) may include a label-accepting site which does not include the label. A first image can then be obtained, and a subsequent treatment carried out to cleave the label attached to the first nucleotide type, and to attach the label to the label-accepting site on the fourth nucleotide type. A second image may then be obtained. For example, the first nucleotide type (e.g. A) may be detected in a channel (e.g. configured to detect the wavelength, such as green light) in the first image and not detected in the channel in the second image, the second nucleotide type (e.g. G) may not be detected in the channel in the first image and may not be detected in the channel in the second image, the third nucleotide type (e.g. T) may be detected in the channel (e.g. configured to detect the wavelength, such as green light) in the first image and may be detected in the channel (e.g. configured to detect the wavelength, such as green light) in the second image, and the fourth nucleotide type (e.g. C) may not be detected in the channel in the first image and may be detected in the channel in the second image (e.g. configured to detect the wavelength, such as green light). Although specific pairings of bases to signal types (e.g. wavelengths) and/or combinations of images are described above, different signal types (e.g. wavelengths), images and/or permutations may also be used.

In one embodiment, the sequencing process comprises a first sequencing read (referred to herein as R1) and second sequencing read (referred to herein as R2). As described below, in each read at least two different polynucleotide strands may be sequenced simultaneously, generating a R1.1 and R1.2 read and a R2.1 and R2.2 read. The first sequencing read and the second sequencing read may also be conducted concurrently. In other words, the first sequencing read and the second sequencing read may be conducted at the same time.

The first sequencing read may comprise the binding of a first sequencing primer (also known as a read 1 sequencing primer) to the first sequencing primer binding site. The second sequencing read may comprise the binding of a second sequencing primer (also known as a read 2 sequencing primer) to the second sequencing primer binding site.

Alternative methods of sequencing include sequencing by ligation, for example as described in U.S. Pat. No. 6,306,597 or WO 06/084132, the contents of which are incorporated herein by reference.

### Data Analysis Using 16 QaM

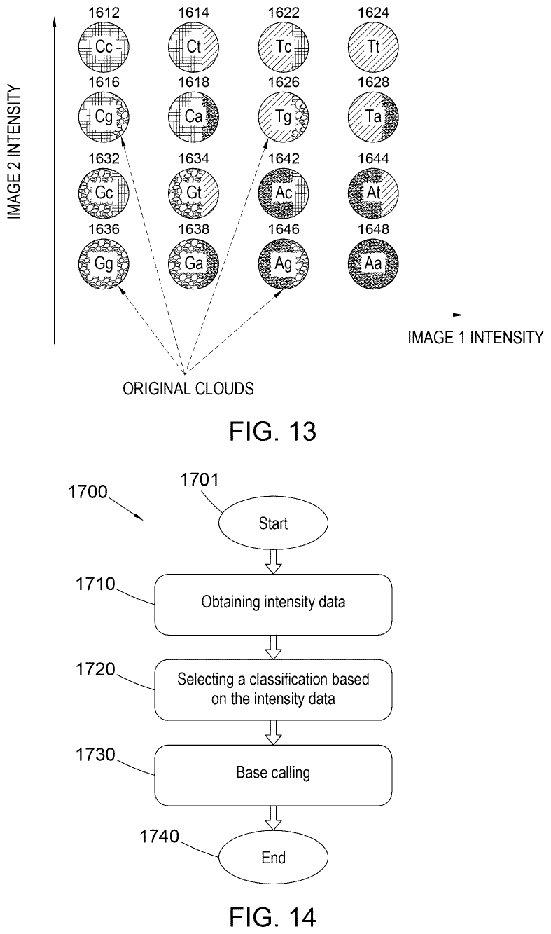

FIG. 13 is a scatter plot showing an example of sixteen distributions of signals generated by polynucleotide sequences disclosed herein.

The scatter plot of FIG. 13 shows sixteen distributions (or bins) of intensity values from the combination of a brighter signal (i.e. a first signal as described herein) and a dimmer signal (i.e. a second signal as described herein); the two signals may be co-localized and may not be optically resolved as described above. The intensity values shown in FIG. 13 may be up to a scale or normalisation factor; the units of the intensity values may be arbitrary or relative (i.e., representing the ratio of the actual intensity to a reference intensity). The sum of the brighter signal generated by the first portions and the dimmer signal generated by the second portions results in a combined signal. The combined signal may be captured by a first optical channel and a second optical channel. Since the brighter signal may be A, T, C or G, and the dimmer signal may be A, T, C or G, there are sixteen possibilities for the combined signal, corresponding to sixteen distinguishable patterns when optically captured. That is, each of the sixteen possibilities corresponds to a bin shown in FIG. 13. The computer system can map the combined signal generated into one of the sixteen bins, and thus determine the added nucleobase at the first portion and the added nucleobase at the second portion, respectively.

For example, when the combined signal is mapped to bin 1612 for a base calling cycle, the computer processor base calls both the added nucleobase at the first portion and the added nucleobase at the second portion as C. When the combined signal is mapped to bin 1614 for the base calling cycle, the processor base calls the added nucleobase at the first portion as C and the added nucleobase at the second portion as T. When the combined signal is mapped to bin 1616 for the base calling cycle, the processor base calls the added nucleobase at the first portion as C and the added nucleobase at the second portion as G. When the combined signal is mapped to bin 1618 for the base calling cycle, the processor base calls the added nucleobase at the first portion as C and the added nucleobase at the second portion as A.

When the combined signal is mapped to bin 1622 for the base calling cycle, the processor base calls the added nucleobase at the first portion as T and the added nucleobase at the second portion as C. When the combined signal is mapped to bin 1624 for the base calling cycle, the processor base calls both the added nucleobase at the first portion and the added nucleobase at the second portion as T. When the combined signal is mapped to bin 1626 for the base calling cycle, the processor base calls the added nucleobase at the first portion as T and the added nucleobase at the second portion as G. When the combined signal is mapped to bin 1628 for the base calling cycle, the processor base calls the added nucleobase at the first portion as T and the added nucleobase at the second portion as A.

When the combined signal is mapped to bin 1632 for the base calling cycle, the processor base calls the added nucleobase at the first portion as G and the added nucleobase at the second portion as C. When the combined signal is mapped to bin 1634 for the base calling cycle, the processor base calls the added nucleobase at the first portion as G and the added nucleobase at the second portion as T. When the combined signal is mapped to bin 1636 for the base calling cycle, the processor base calls both the added nucleobase at the first portion and the added nucleobase at the second portion as G. When the combined signal is mapped to bin 1638 for the base calling cycle, the processor base calls the added nucleobase at the first portion as G and the added nucleobase at the second portion as A.

When the combined signal is mapped to bin 1642 for the base calling cycle, the processor base calls the added nucleobase at the first portion as A and the added nucleobase at the second portion as C. When the combined signal is mapped to bin 1644 for the base calling cycle, the processor base calls the added nucleobase at the first portion as A and the added nucleobase at the second portion as T. When the combined signal is mapped to bin 1646 for the base calling cycle, the processor base calls the added nucleobase at the first portion as A and the added nucleobase at the second portion as G. When the combined signal is mapped to bin 1648 for the base calling cycle, the processor base calls both the added nucleobase at the first portion and the added nucleobase at the second portion as A.

In this particular example, T is configured to emit a signal in both the IMAGE 1 channel and the IMAGE 2 channel, A is configured to emit a signal in the IMAGE 1 channel only, C is configured to emit a signal in the IMAGE 2 channel only, and G does not emit a signal in either channel. However, different permutations of nucleobases can be used to achieve the same effect by performing dye swaps. For example, A may be configured to emit a signal in both the IMAGE 1 channel and the IMAGE 2 channel, T may be configured to emit a signal in the IMAGE 1 channel only, C may be configured to emit a signal in the IMAGE 2 channel only, and G may be configured to not emit a signal in either channel.

Further details regarding performing base-calling based on a scatter plot having sixteen bins may be found in U.S. Patent Application Publication No. 2019/0212294, the disclosure of which is incorporated herein by reference.

FIG. 14 is a flow diagram showing a method 1700 of base calling according to the present disclosure. The described method allows for simultaneous sequencing of two (or more) portions (e.g. the first portion and the second portion) in a single sequencing run from a single combined signal obtained from the first portion and the second portion, thus requiring less sequencing reagent consumption and faster generation of data from both the first portion and the second portion. Further, the simplified method may reduce the number of workflow steps while producing the same yield as compared to existing next-generation sequencing methods. Thus, the simplified method may result in reduced sequencing runtime.

As shown in FIG. 14, the disclosed method 1700 may start from block 1701. The method may then move to block 1710.

At block 1710, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data that corresponds to two (or more) different portions (e.g. the first portion and the second portion). In one example, intensity data is selected based upon a chastity score. A chastity score may be calculated as the ratio of the brightest base intensity divided by the sum of the brightest and second brightest base intensities. The desired chastity score may be different depending upon the expected intensity ratio of the light emissions associated with the different portions. As described above, it may be desired to produce clusters comprising the first portion and the second portion, which give rise to signals in a ratio of 2:1. In one example, high-quality data corresponding to two portions with an intensity ratio of 2:1 may have a chastity score of around 0.8 to 0.9.

After the intensity data has been obtained, the method may proceed to block 1720. In this step, one of a plurality of classifications is selected based on the intensity data. Each classification represents a possible combination of respective first and second nucleobases. In one example, the plurality of classifications comprises sixteen classifications as shown in FIG. 13, each representing a unique combination of first and second nucleobases. Where there are two portions, there are sixteen possible combinations of first and second nucleobases. Selecting the classification based on the first and second intensity data comprises selecting the classification based on the combined intensity of the first and second signal components and the combined intensity of the third and fourth signal components.

The method may then proceed to block 1730, where the respective first and second nucleobases are base called based on the classification selected in block 1720. The signals generated during a cycle of a sequencing are indicative of the identity of the nucleobase(s) added during sequencing (e.g. using sequencing-by-synthesis). It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1740.

### Data Analysis Using 9 QaM

For two portions of polynucleotide sequences (e.g. a first portion and a second portion as described herein), there are sixteen possible combinations of nucleobases at any given position (i.e., an A in the first portion and an A in the second portion, an A in the first portion and a T in the second portion, and so on). When the same nucleobase is present at a given position in both portions, the light emissions associated with each target sequence during the relevant base calling cycle will be characteristic of the same nucleobase. In effect, the two portions behave as a single portion, and the identity of the bases at that position are uniquely callable.

However, when a nucleobase of the first portion is different from a nucleobase at a corresponding position of the second portion, the signals associated with each portion in the relevant base calling cycle will be characteristic of different nucleobases. In one embodiment, the first signal coming from the first portion have substantially the same intensity as the second signal coming from the second portion. The two signals may also be co-localised, and may not be spatially and/or optically resolved. Therefore, when different nucleobases are present at corresponding positions of the two portions, the identity of the nucleobases cannot be uniquely called from the combined signal alone. However, useful sequencing information can still be determined from these signals.

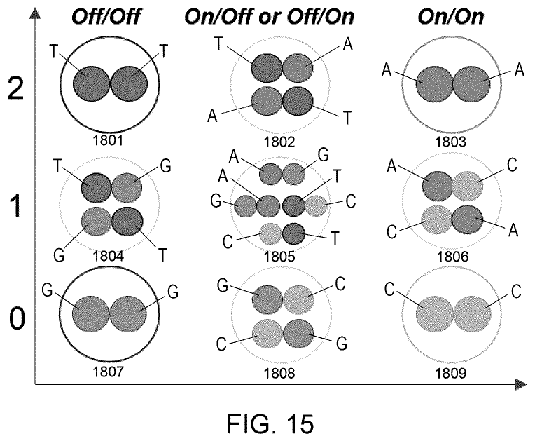

The scatter plot of FIG. 15 shows nine distributions (or bins) of intensity values from the combination of two co-localised signals of substantially equal intensity.

The intensity values shown in FIG. 15 may be up to a scale or normalisation factor; the units of the intensity values may be arbitrary or relative (i.e., representing the ratio of the actual intensity to a reference intensity). The sum of the first signal generated from the first portion and the second signal generated from the second portion results in a combined signal. The combined signal may be captured by a first optical channel and a second optical channel. The computer system can map the combined signal generated into one of the nine bins, and thus determine sequence information relating to the added nucleobase at the first portion and the added nucleobase at the second portion.

Bins are selected based upon the combined intensity of the signals originating from each target sequence during the base calling cycle. For example, bin 1803 may be selected following the detection of a high-intensity (or “on/on”) signal in the first channel and a high-intensity signal in the second channel. Bin 1806 may be selected following the detection of a high-intensity signal in the first channel and an intermediate-intensity (“on/off” or “off/on”) signal in the second channel. Bin 1809 may be selected following the detection of a high-intensity signal in the first channel and a low-intensity or zero-intensity (“off/off”) signal in the second channel. Bin 1802 may be selected following the detection of an intermediate-intensity signal in the first channel and a high-intensity signal in the second channel. Bin 1805 may be selected following the detection of an intermediate-intensity signal in the first channel and an intermediate-intensity signal in the second channel. Bin 1808 may be selected following the detection of an intermediate-intensity signal in the first channel and a low-intensity or zero-intensity signal in the second channel. Bin 1801 may be selected following the detection of a low-intensity signal in the first channel and a high-intensity signal in the second channel. Bin 1804 may be selected following the detection of a low-intensity or zero-intensity signal in the first channel and an intermediate-intensity signal in the second channel. Bin 1807 may be selected following the detection of a low-intensity or zero-intensity signal in the first channel and a low-intensity signal in the second channel.

Four of the nine bins represent matches between respective nucleobases of the two portions sensed during the cycle (bins 1801, 1803, 1807, and 1809). In response to mapping the combined signal to a bin representing a match, the computer processor may detect a match between the first portion and the second portion at the sensed position. In response to mapping the combined signal to a bin representing a match, the computer processor may base call the respective nucleobases. For example, when the combined signal is mapped to bin 1801 for a base calling cycle, the computer processor base calls both the added nucleobase at the first portion and the added nucleobase at the second portion as T. When the combined signal is mapped to bin 1803 for the base calling cycle, the processor base calls both the added nucleobase at the first portion and the added nucleobase at the second portion as A. When the combined signal is mapped to bin 1807 for the base calling cycle, the processor base calls both the added nucleobase at the first portion and the added nucleobase at the second portion as G. When the combined signal is mapped to bin 1809 for the base calling cycle, the processor base calls both the added nucleobase at the first portion and the added nucleobase at the second portion as C.

The remaining five bins are “ambiguous”. That is to say that these bins each represent more than one possible combination of first and second nucleobases. Bins 1802, 1804, 1806, and 1808 each represent two possible combinations of first and second nucleobases. Bin 1805, meanwhile, represents four possible combinations. Nevertheless, mapping the combined signal to an ambiguous bin may still allow for sequencing information to be determined. For example, bins 1802, 1804, 1805, 1806, and 1808 represent mismatches between respective nucleobases of the two portions sensed during the cycle. Therefore, in response to mapping the combined signal to a bin representing a mismatch, the computer processor may detect a mismatch between the first portion and the second portion at the sensed position.

In this particular example, A is configured to emit a signal in both the first channel and the second channel, C is configured to emit a signal in the first channel only, T is configured to emit a signal in the second channel only, and G does not emit a signal in either channel. However, different permutations of nucleobases can be used to achieve the same effect by performing dye swaps. For example, A may be configured to emit a signal in both the first channel and the second channel, T may be configured to emit a signal in the first channel only, C may be configured to emit a signal in the second channel only, and G may be configured to not emit a signal in either channel.

The number of classifications, which may be selected based upon the combined signal intensities may be predetermined, for example based on the number of portions expected to be present in the nucleic acid cluster. Whilst FIG. 15 shows a set of nine possible classifications, the number of classifications may be greater or smaller.

In addition to identifying matches and mismatches, the mapping of the combined signal to each of the different bins (e.g. in combination with additional knowledge, such as the library preparation methods used) can provide additional information about the first portion and the second portion, or about sequences from which the first portion and the second portion were derived. For example, given the nucleic acid material input and the processing methods used to generate the nucleic acid clusters, the first portion and the second portion may be expected to be identical at a given position. In this case, the mapping of the combined signal to a bin representing a mismatch may be indicative of an error introduced during library preparation. In addition, the first portion and the second portion may be expected to be different, for example due to deliberate sequence modifications introduced during library preparation to detect modified cytosines.

Errors arise during NGS library preparation, for example due to PCR artefacts or DNA damage. The error rate is determined by the library preparation method used, for example the number of cycles of PCR amplification carried out, and a typical error rate may be of the order of 0.1%. This limits the sensitivity of diagnostic assays based on the sequencing method, and may obscure true variants. The present methods allow for the identification of library preparation errors from fewer sequencing reads.

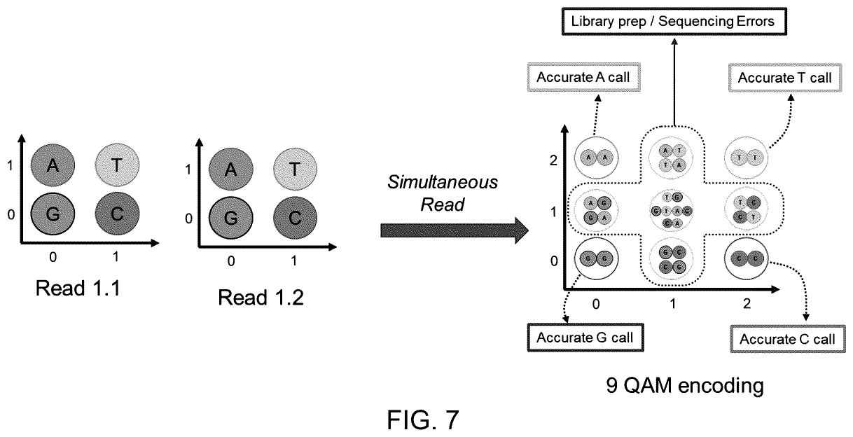

In the absence of any library preparation/sequencing errors, the signals produced by sequencing the two portions (e.g. using sequencing-by-synthesis) will match. The combined signal may therefore be mapped to one of the four “corner” clouds shown in FIGS. 7 and 8, and FIG. 15, and the identity of the nucleobase at the corresponding position of the original library polynucleotide can be determined. Should the identity of the nucleobase at that position suggest a rare, or even unknown, variant, it can be determined with a high level of confidence that the base call represents a true variant, as opposed to a library preparation error. If, on the other hand, the combined signal is mapped to any of the other clouds, this indicates that the sequences of the first portion and the second portion do not match, and that an error has occurred in library preparation. Therefore, in response to mapping the combined signal to a classification representing a mismatch between the two nucleobases, a library preparation error may be identified.

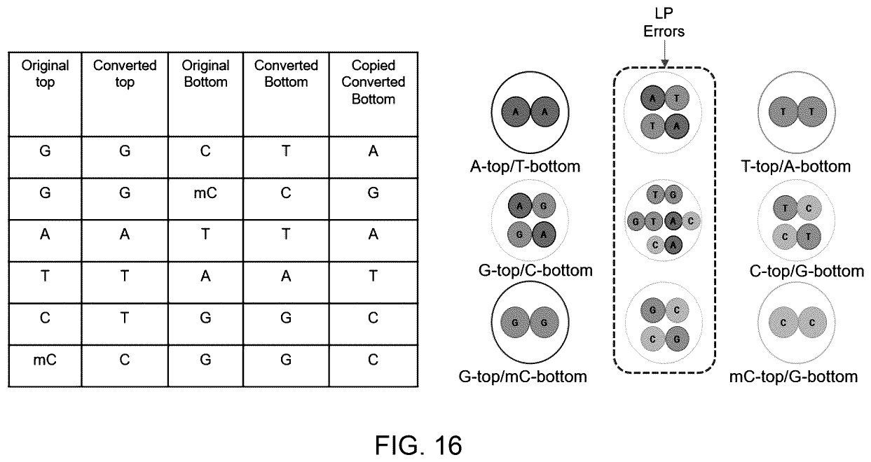

As mentioned herein, the library preparation may involve treatment with a conversion agent. In cases where the conversion reagent is configured to convert an unmodified cytosine to uracil or a nucleobase which is read as thymine/uracil, the correspondence between bases in the original polynucleotide and in the converted strands is shown in FIG. 16, alongside a scatter plot showing potential resulting distributions for the combined signal intensities resulting from the simultaneous sequencing of the target sequences. An A-T or T-A base pair in the original molecule will result in a match (A/A or T/T) at the corresponding position of the forward and reverse complement strands of the library. An mC-G or G-mC base pair in the library will also result in a match (G/G or C/C) at the corresponding position of the forward and reverse complement strands of the library. For a C-G base pair, however, the conversion of unmodified cytosine to uracil (or a nucleobase which is read as thymine/uracil) in the forward strand of the library (“top” strand) will result in a T at the corresponding position of the forward strand of the library. Meanwhile, the corresponding position on the reverse complement strand of the library (“bottom” strand) will be occupied by C. Alternatively, for a G-C base pair, the conversion of unmodified cytosine to uracil (or a nucleobase which is read as thymine/uracil) in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing G/G or C/C, the presence of a modified cytosine can be determined at the corresponding position in the original polynucleotide.

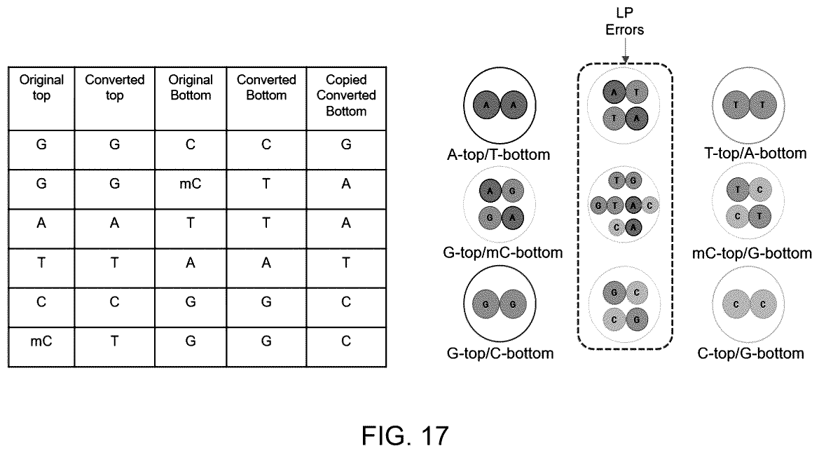

In other cases where the conversion reagent is configured to convert a modified cytosine to thymine or a nucleobase which is read as thymine/uracil, FIG. 17 shows the correspondence between bases in the original polynucleotide and in the converted strands, alongside a scatter plot showing potential resulting distributions for the combined signal intensities resulting from the simultaneous sequencing of the target sequences. An A-T or T-A base pair in the library will result in a match (A/A or T/T) at the corresponding position of the forward and reverse complement strands of the library. A C-G or G-C base pair in the library will also result in a match (G/G or C/C) at the corresponding position of the forward and reverse complement strands of the library. For a mC-G base pair, however, the conversion of 5-methylcytosine to thymine in the forward strand of the library (“top” strand) will result in a T at the corresponding position of the forward strand of the library. Meanwhile, the corresponding position on the reverse complement strand of the library (“bottom” strand) will be occupied by C. Alternatively, the conversion of 5-methylcytosine to thymine in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing an A/G, G/A, T/C, or C/T mismatch, the presence of a modified cytosine can be determined at the corresponding position in the original polynucleotide.

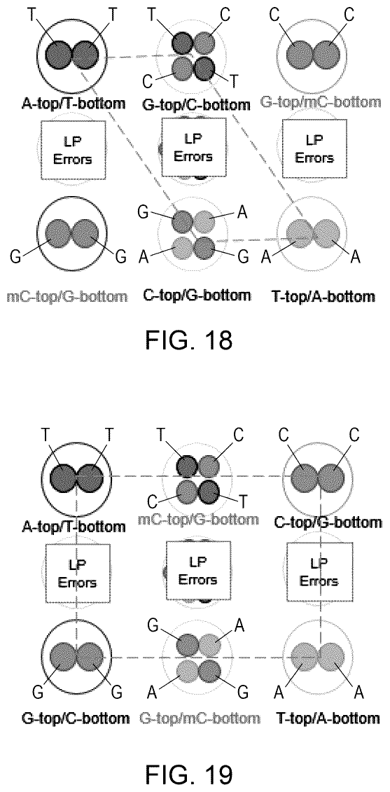

FIG. 18 represents the distributions resulting from the use of an alternative dye-encoding scheme following use of a conversion reagent configured to convert an unmodified cytosine to uracil or a nucleobase which is read as thymine/uracil, and FIG. 19 represents the distributions resulting from the use of an alternative dye-encoding scheme following use of a conversion reagent configured to convert a modified cytosine to thymine or a nucleobase which is read as thymine/uracil.

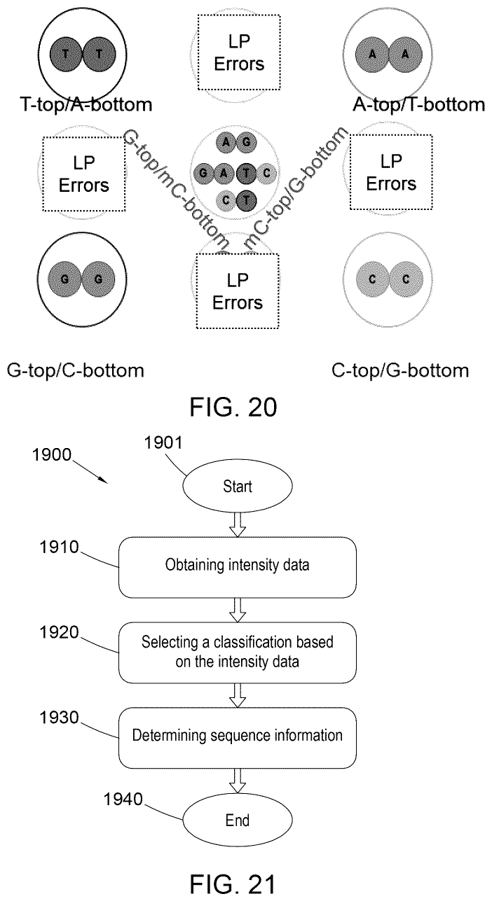

FIG. 20 represents yet another distribution resulting from the use of an alternative dye-encoding scheme following use of a conversion reagent configured to convert a modified cytosine to thymine or a nucleobase, which is read as thymine/uracil. In this case, modified cytosines fall within a central bin.

In the present example, for each base pair in the original double-stranded DNA molecule, it may be assumed that there are six possibilities: A-T, T-A, C-G, G-C, mC-G and G-mC. As shown in FIGS. 16 to 19, each of these possibilities is uniquely represented by one of the plurality of classifications. According to the present methods, it is therefore possible to determine both the sequence and “methylation” status (i.e. presence of modified cytosines) of a double-stranded polynucleotide in a single sequencing run.

In addition to determining “methylation” status, it may also be possible to identify library preparation/sequencing errors. Using the dye-encoding scheme shown in FIGS. 16 and 17, the central column of distributions is indicative of such errors. Using the dye encoding scheme shown in FIGS. 18 and 19, the central row of distributions is indicative of such errors.

The dye-encoding scheme may be optimised to allow for different combinations of first and second nucleobases to be resolved. This may be particularly useful where sequence modifications of a known type have been introduced into the first portions and the second portions. For example, where sequence modifications have been introduced that result in the conversion of unmodified cytosines to uracil or nucleobases which is read as thymine/uracil, or the conversion of modified cytosines to thymine or nucleobases which are read as thymine/uracil, the dye-encoding scheme may be selected such that the resulting combination of first and second nucleobases do not fall within the central bin (which represents four different nucleobase combinations).

In the case of conversion of modified cytosines to thymine (or nucleobases which are read as thymine/uracil), a T/C or G/A mismatch between the forward and reverse complement strands is indicative of the presence of a mC-G or G-mC base pair at the corresponding position of the library. The dye-encoding scheme may therefore be designed such that these mismatches may be resolved from other possible combinations of nucleobases. This may be achieved by detecting light emissions from A and T bases in a first illumination cycle, and from C and T bases in a second illumination cycle. In another example, light emissions may be detected from C and G bases in a first illumination cycle, and from C and T bases in a second illumination cycle. In another example, light emissions may be detected from C and A bases in a first illumination cycle, and from C and G bases in a second illumination cycle.

In the case of unmodified cytosines to uracil (or nucleobases which is read as thymine/uracil), a C/C or G/G match between the forward and reverse complement strands is indicative of the presence of a mC-G or G-mC base pair at the corresponding position of the library. In this case, a mC-G or G-mC base pair will always be resolvable. However, the dye-encoding scheme can still be designed to optimise the resolution between unmodified bases.

FIG. 21 is a flow diagram showing a method 1900 of determining sequence information according to the present disclosure. The described method allows for the determination of sequence information from two (or more) portions (e.g. the first portion and the second portion) in a single sequencing run from a single combined signal obtained from the first portion and the second portion.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 or at least 50 base pairs and the second portion is at least 25 base pairs or at least 50 base pairs.

As shown in FIG. 21, the disclosed method 1900 may start from block 1901. The method may then move to block 1910.

At block 1910, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data, for example based upon a chastity score. A chastity score may be calculated as the ratio of the brightest base intensity divided by the sum of the brightest and second brightest base intensities. In one example, high-quality data corresponding to two portions with a substantially equal intensity ratio may have a chastity score of around 0.8 to 0.9, for example 0.89-0.9.

After the intensity data has been obtained, the method may proceed to block 1920. In this step, one of a plurality of classifications is selected based on the intensity data. Each classification represents one or more possible combinations of respective first and second nucleobases, and at least one classification of the plurality of classifications represents more than one possible combination of respective first and second nucleobases. In one example, the plurality of classifications comprises nine classifications as shown in FIG. 15. Selecting the classification based on the first and second intensity data comprises selecting the classification based on the combined intensity of the first and second signal components and the combined intensity of the third and fourth signal components.

The method may then proceed to block 1930, where sequence information of the respective first and second nucleobases is determined based on the classification selected in block 1920. The signals generated during a cycle of a sequencing are indicative of the identity of the nucleobase(s) added during sequencing (e.g. using sequencing-by-synthesis). For example, it may be determined that there is a match or a mismatch between the respective first and second nucleobases. Where it is determined that there is a match between the first and second respective nucleobases, the nucleobases may be base called. Whether there is a match or a mismatch, additional or alternative information may be obtained, as described above. It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1940.

### Methods of Preparing and Sequencing a Tandem Library

In one aspect of the invention, there is provided a method of preparing at least one polynucleotide library strand, wherein the method comprises:
attaching a first adaptor to a first end of a double-stranded polynucleotide sequence, wherein the first end comprises the 3′ end of the forward strand and the 5′ end of the reverse strand of the double-stranded polynucleotide sequence; and
attaching a second adaptor to a second end of a double-stranded polynucleotide sequence, wherein the second end comprises the 5′ end of the forward strand and the 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease.

In another aspect of the invention, there is provided a method of preparing at least one polynucleotide library strand, wherein the method comprises:
attaching a first adaptor to a first end of a double-stranded polynucleotide sequence, wherein the first end comprises the 3′ end of the forward strand and the 5′ end of the reverse strand of the double-stranded polynucleotide sequence; and
attaching a second adaptor to a second end of a double-stranded polynucleotide sequence, wherein the second end comprises the 5′ end of the forward strand and the 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the second adaptor comprises a cleavable site and/or a complement of a cleavable site.

In another aspect of the invention, there is provided a method of preparing at least one polynucleotide library strand, wherein the method comprises:
attaching a first adaptor to a first end of a double-stranded polynucleotide sequence, wherein the first end comprises the 3′ end of the forward strand and the 5′ end of the reverse strand of the double-stranded polynucleotide sequence; and
attaching a second adaptor to a second end of a double-stranded polynucleotide sequence, wherein the second end comprises the 5′ end of the forward strand and the 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease and wherein the second adaptor comprises a cleavable site and/or a complement of a cleavable site.

In another aspect of the invention, there is provided a polynucleotide library strand for sequencing comprising a first adaptor, a double-stranded polynucleotide sequence to be identified and a second adaptor, wherein the first adaptor is attached to a first end of the double-stranded polynucleotide sequence, wherein the first end comprises the 3′ end of the forward strand and the 5′ end of the reverse strand of the double-stranded polynucleotide sequence; and the second adaptor is attached to a second end of the double-stranded polynucleotide sequence, wherein the second end comprises the 5′ end of the forward strand and the 3′ end of the reverse strand of the double-stranded polynucleotide sequence; wherein the first adaptor comprises a loop that connects the 3′ end of the forward strand and the 5′ end of the reverse strand, and wherein the second adaptor comprises a base-paired stem, a primer-binding complement sequence and a primer-binding sequence, and wherein the first adaptor comprises at least one restriction site for an endonuclease.

The first and second adaptors may be attached to the polynucleotide using processes as described in more detail in e.g. WO 07/052006, or “tagmentation” methods as described above.

In a further embodiment, the second adaptor may also comprise at least one cleavable site. In other words, the first adaptor comprises at least one restriction site and the second adaptor comprises at least one cleavable site. The cleavable site may also be a restriction site.

By “restriction site” is meant a sequence of nucleotides recognised by an endonuclease, such as a single-stranded endonuclease. A restriction site may also be referred to as a “recognition site” or “recognition sequence”, and such terms may be used interchangeably.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb.BbvCI, Nb.Bsml, Nb.BsrDI, Nb.Btsl, Nt.Alwl, Nt.BsmAl, Nt.BspQI, Nt.BstNBI, BssSI, Nb.Bpu101 and Nt.CviPII. These nickases can be used either alone or in various combinations. Other suitable nicking endonucleases are available from commercial sources, including New England Biolabs and Fisher Scientific.

The restriction sites vary depending on the nickase used, and are well known in the art. In one example, the restriction site is selected from the following:

In one embodiment, the nickase is Nb.BssSI, and the restriction site is CACGAG, wherein Nb.BssSI catalyzes a single strand break within the recognition sequence.

In one embodiment, the nickase is Nt.BspQI, and the restriction site is GCTCTTC (1/−7), wherein Nt.BspQI catalyzes a single strand break one base beyond the 3′ side of the restriction site.

In one embodiment, the nickase is Nt.CviPII and the restriction site is (0/−1) CCD, wherein Nt.CviPII catalyzes a single strand break at the 5′ side of the restriction site.

In one embodiment, the nickase is Nt.BstNBI and the restriction site is GAGTC (4/−5), wherein Nt.BstNBI catalyzes a single strand break four bases beyond the 3′ side of the restriction site.

In one embodiment, the nickase is Nb.BsrDI and the restriction site is GCAATG, wherein Nb.BsrDI catalyzes a single strand break within the restriction site.

In one embodiment, the nickase is Nb.Btsl and the restriction site is GCAGTG, wherein Nb.Btsl catalyzes a single strand break within the restriction site.

In one embodiment, the nickase is Nt.Alwl and the restriction site is GGATC (4/−5), wherein Nt.Alwl catalyzes a single strand break four bases beyond the 3′ side of the restriction site.

In one embodiment, the nickase is Nb.BbvCI and the restriction site is CCTCAGC, wherein Nb. BbvCI catalyzes a single strand break within the restriction site.

In one embodiment, the nickase is Nb.Bsml and the restriction site is GAATGC, wherein Nb.Bsml catalyzes a single strand break within the restriction site.

In one embodiment, the nickase is Nt.BsmAl and the restriction site is GTCTC (1/−5), wherein Nt. BsmAl catalyzes a single strand break one base beyond the 3′ side of the restriction site.

In one embodiment, the nickase is Nb.Bpu101 and the restriction site is CCTNAGC, wherein Nb.Bpu101 catalyzes a single strand break within the restriction site.

Where the restriction site is described in the following format (x/−y), x is the number of nucleotides beyond (i.e. 3′ of) the 3′ end of the restriction site where cleavage occurs; and y is the number of nucleotides in the restriction site.

In an alternative embodiment, the endonuclease is a Cas9 nickase.

Examples of a Cas9 nickase include Cas9 D10A and Cas9 H840A. For example, in one embodiment, the Cas9 protein may comprise the D10A or H840A amino acid substitutions. These nickases cleave only the DNA strand that is complementary to and recognized by a gRNA.

In one embodiment, the restriction site may be or may comprise a PAM (protospacer adjacent motif) sequence. Examples of suitable PAM sequences include NGG, NGAG, NGCG, NGN, NG, GAA, GAT, NNG, NGN, NRN, YG, NNGRRT, NNNRRT, NNAGAA, NNNNGATT and NNNNCRAA and complements thereof.

In a further embodiment, the Cas9 protein may alternatively or additionally comprise the N863A or N854A amino acid substitutions.

In a further embodiment, the Cas9 protein has been modified to improve activity. For example, in one embodiment, the Cas9 protein may additionally comprise a D1135E substitution. Alternatively, the Cas9 protein may also be the VQR variant.

In one embodiment, where the first and second adaptors both comprise a restriction site, the restriction sites are different sequences. Accordingly, in one embodiment, the first adaptor comprises a first restriction site and the second adaptor comprises a second restriction site.

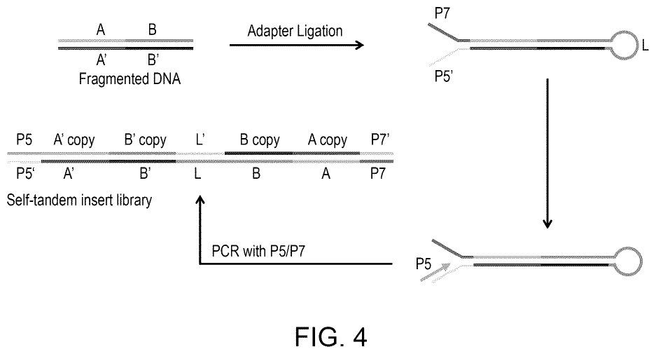

In one embodiment, the target polynucleotide to be sequenced is a double stranded polynucleotide molecule (also referred to herein as a duplex), for example, as shown in FIG. 4. Accordingly, the target polynucleotide may be considered to have a first portion to be identified and a second portion to be identified, wherein the first portion is the forward strand and wherein the second portion is the reverse strand. As shown in FIG. 4, A represents the 5′ “half” of the forward strand and B represents the 3′ “half” of the forward strand. Similarly, A′ represents the complement of the 5′ “half” of the forward strand (i.e. it is the 3′ “half” of the reverse strand) and B′ represents the complement of the 3′ “half” of the forward strand (i.e. it is the 5′ “half” of the reverse strand.

The first adaptor may be attached to the 5′ end of the first portion and the 3′ end of the second portion. Similarly, the second adaptor may be attached to the 3′ end of the first portion and the 5′ end of the second portion.

In one embodiment, the first adaptor is added to the 3′ end of the polynucleotide duplex (that is, the 3′ end of the forward strand and the 5′ end of the reverse strand). The first adaptor may be an oligonucleotide of any structure or any sequence that allows the forward and reverse strands to be connected. For example, the adaptor may be capable of forming a loop. In one example, as shown in FIG. 4, the first adaptor comprises a base-paired stem and a hairpin loop (e.g. a loop structure with unpaired or non-Watson-Crick paired nucleotides) and connects the 3′ end of the forward strand with the 5′ end of the reverse strand.

In one embodiment, the (first) restriction site is in the base-paired stem, at either the 5′ or 3′ end of the base-paired stem. In one aspect, the restriction site is at the 5′ end.

Where the first adaptor comprises a first restriction site, the location of the restriction sequence will depend on whether the cleavage site for the target endonuclease is immediately 3′ of the restriction site or whether, as described above, the endonuclease cleaves (nicks) a number of nucleotides 3′ of the restriction site. It is of course desirable that the endonuclease does not cleave in the target polynucleotide to be sequenced or in its complement on the template (i.e. in the first or second portions, which are the portions that allow the target polynucleotide to be sequenced).

In one embodiment, the second adaptor comprises at least one primer-binding sequence. In another embodiment, the second adaptor comprises at least one primer-binding complement sequence. In an alternative embodiment, the second adaptor comprises both a primer-binding sequence and a primer-binding complement sequence. The primer-binding sequence may be capable of binding to a lawn or immobilised primer that is immobilised on the surface of a solid support. For example, the primer-binding sequence may be either P5′ (for example, SEQ ID NO: 3 or a variant or fragment thereof) or P7′ (for example, SEQ ID NO: 4 or a variant or fragment thereof). Similarly, the primer-binding complement sequence may be either P5 (for example, SEQ ID NO: 1 or 5 or a variant or fragment thereof) or P7 (for example, SEQ ID NO: 2 or a variant or fragment thereof). If the primer-binding sequence is P5′, the primer-binding complement sequence is P7. If the primer-binding sequence is P7′, the primer-binding complement sequence is P5.

As shown in FIG. 4, the second adaptor comprises a base-paired stem, a primer-binding sequence and a primer-binding complement sequence. Specifically, the second adaptor may comprise a first and second strand, wherein the first and second strands are base-paired for a portion of their sequence (forming the base-paired stem) and are non-complementary for the remainder of their sequence, for example, P5′ and P7 or P7′ and P5, which subsequently forms a fork structure, wherein a first arm of the fork structure comprises a primer-binding sequence and the second arm of the fork structure comprises a primer-binding complement sequence.

In one embodiment the second adaptor comprises a (first) cleavable site. In one embodiment, the cleavable site is in the base-paired stem. As described above, the base-paired stem comprises two strands. In one example, the first strand comprises a cleavable site and the second strand comprises a complement of the cleavable site. In one embodiment, it is the strand that is attached to the primer-binding complement sequence that comprises the cleavable site, and the strand that is attached to the primer-binding sequence that comprises a complement of the cleavable site. The cleavable site and the complement of the cleavable site may be cleavable by the same cleaving agent (i.e. they are complementary sequences), although it is possible for the sequences to be cleavable by different agents (i.e. they are not complementary sequences of each other).

Alternatively, the second adaptor does not comprise a cleavable site in the base-paired stem.

In another embodiment, the second adaptor comprises a base-paired stem and a first arm of a fork and a second arm of a fork, where the first arm comprises a primer-binding sequence and a complement of a cleavable site, and the second arm comprises a primer-binding complement sequence and a cleavable site. Again, the cleavable site and complement thereof may be cleavable by the same cleaving agent or different cleaving agents, as described above.

Alternatively, the second adaptor may comprise a base-paired stem and a hairpin loop, where the loop comprises a primer-binding sequence, a second cleavable site and primer-binding complement sequence, where the cleavable site is in-between the primer-binding sequence and the primer-binding complement sequence. In one embodiment, the first adaptor comprises a first cleavable site in the base-paired stem as described above, and a second cleavable site in the loop and in-between the primer-binding sequence and the primer-binding complement sequence. Alternatively, the second adaptor does not comprise the first cleavable site.

As used herein, by “cleavable site” is meant any moiety, such as a modified nucleotide, that allows selective cleavage of the adaptor sequence. By way of non-limiting example, the cleavable site may comprise uracil bases, phosphorothioate groups, ribonucleotides, diol linkages, disulphide linkages, peptides etc.

In one example, the cleavable site is a uracil. Uracil can be cleaved using a uracil glycosylase or USER enzyme mix (which is a cocktail of uracil glycosylase and endonuclease VIII).

In another example, the cleavable site is 8-oxoguanine. 8-oxoguanine can be cleaved using a FPG glycosylase.

Alternatively, the cleavable site is a restriction site. In one embodiment, the first cleavable site is a restriction site. As referred to herein the first cleavable site may therefore be referred to as the second restriction site, and the second cleavable site may be referred to herein as the third restriction site. In some embodiments, the first, second and third restriction sites are all different (i.e. different restriction site sequences).

In one embodiment, the method may comprise cleaving the loop of the second adaptor at the cleavable site to open the loop. This will generate a fork structure, as described above. Specifically, following cleavage the second adaptor will form a base-paired stem and then a fork.

Although not shown in FIG. 4, the first and second adaptors also comprise one or more sequencing primer-binding sites and/or sequencing primer-binding sites. Both are referred to generally as primer-binding sites.

In the first adaptor the sequencing primer-binding sites may be in the loop sequence or in the base-paired stem. In one embodiment, the base-paired stem comprises at least one sequencing primer-binding site. In one embodiment, the sequencing primer-binding site is in the base-paired stem, and in the part of the stem that connects to the reverse strand of the double-stranded polynucleotide. In another embodiment, the loop may comprise two sequencing primer sites. In one example, the loop comprises two sequencing primer sites and a restriction site, wherein the sequencing primer sites are either side of the restriction site.

In the second adaptor the sequencing primer-binding site(s) may also be in the base-paired stem. Alternatively, each fork of the second adaptor may additionally comprise a sequencing primer-binding site.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The sequence of the sequencing primers and the sequence primer binding sites are not material to the methods of the invention, as long as the sequencing primers are able to bind to the sequence primer binding site to enable amplification and sequencing of the regions to be identified.

In a further embodiment, as also not shown in FIG. 4, the first and/or second adaptors may further comprise one or more index sequences (or one or more index sequence complements).

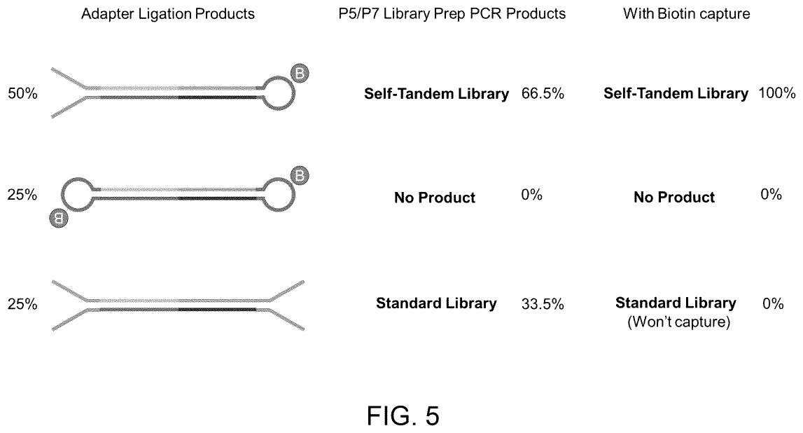

As shown in FIG. 5, after ligation of the adapters three configurations will result, one of which represents the desired loop/fork configuration. The loop/loop configuration does not contain any primer binding sites and will therefore be automatically eliminated during PCR and/or clustering steps. The fork/fork configuration, however, poses an inefficiency risk to the process.

Accordingly, in one embodiment, the first adaptor comprises at least one affinity tag. As such, where required, unwanted fork/fork molecules could easily be eliminated from the workflow via a single affinity-based purification system. As such, the affinity tag may be any tag that can be used in this system. Examples include, but are not limited to, biotin, avidins (e.g. streptavidin), antibodies, haptens, cucubiturils, adamantanes (e.g. 1-adamantylamine), ammonium ions (e.g. amino acids), ferrocenes, cyclodextrins, calixarenes, crown ethers (e.g. 18-crown-6, 15-crown-5, 12-crown-4), cryptands (e.g. [2.2.2]cryptand), His tags (e.g. His₆ tag), or the like.

In one embodiment, the affinity tag is biotin. This would enable the elimination of fork/fork molecules using streptavidin beads (e.g. magnetic streptavidin beads) before/after PCR (FIG. 5). Accordingly, in a further embodiment of the method, the method comprises eliminating polynucleotide library strands with a second adaptor attached to a first end and a second adaptor attached to a second end.

In one embodiment, the method may comprise preparing a polynucleotide library strand as described above, and applying an epigenetic conversion strategy. Such conversion strategies involve treating the polynucleotide library strand with a conversion reagent, wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil. Suitable strategies are well appreciated by the skilled person. Non-limiting examples of such conversion strategies include bisulfite sequencing (BS-seq), oxidative bisulfite sequencing (oxBS-seq), reduced bisulfite sequencing (redBS-seq), TET-assisted bisulfite sequencing (TAB-seq), APOBEC-coupled epigenetic sequencing (ACE-seq), Enzymatic Methyl sequencing (EM-seq), TET-assisted pyridine borane sequencing (TAPS), TET-assisted pyridine borand sequencing with β-glucosyltransferase blocking (TAPSB), chemical-assisted pyridine borane sequencing (CAPS), pyridine borane sequencing (PS), and pyridine borane sequencing for 5-caC (PS-c). Non-limiting examples of conversion reagents include sulfites (e.g. bisulfite), cytidine deaminases (e.g. wild-type or mutant enzymes of the APOBEC family), and boron-based reducing agents (e.g. amine-borane compounds or azine-borane compounds, such as t-butylamine borane, ammonia borane, ethylenediamine borane, dimethylamine borane, pyridine borane and 2-picoline borane),

As used herein, the term “modified cytosine” may refer to any one or more of 5-methylcytosine (5-mC), 5-hydroxymethylcytosine (5-hmC), 5-formylcytosine (5-fC) and 5-carboxylcytosine (5-caC):

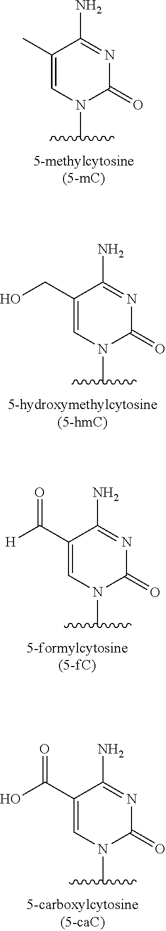

wherein the wavy line indicates an attachment point of the modified cytosine to the polynucleotide.

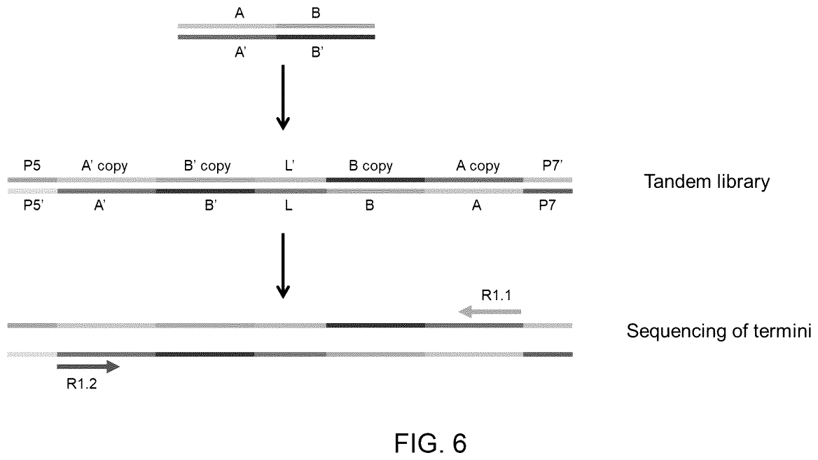

The resulting libraries may either be further amplified via PCR or be directly used for clustering in PCR-free workflows. If amplified, the resulting amplified (double-stranded) library strand is shown in FIG. 6.

As shown in FIG. 6, following binding of a primer (e.g. an immobilised lawn primer, for example P7 (but this could be P5 depending on the arrangement of the forked adaptors)) to a primer-binding sequence (for example, P7′ (but this could be P5′ depending on the arrangement of the forked adaptors)) the library strand can be amplified. Following the first round of amplification, the resulting double-stranded polynucleotide library strands generated from the original library fragment will comprise a forward strand, which corresponds to a complement of the original library fragment (including a complement of the restriction sites) and a reverse strand, which corresponds to the original library fragment.

Accordingly, the forward strand of the resulting amplified library strand will comprise (in the 5′ to 3′ direction):
a complement of a first strand of the first adaptor (comprising a primer-binding complement sequence (e.g. P5, for example, SEQ ID NO: 1 or 5 or a variant or fragment thereof) and a complement of the first strand of the base-paired stem);
a copy of the 3′ end of the reverse strand (of the original library fragment) (A′ copy);
a copy of the 5′ end of the reverse strand (of the original library fragment) (B′ copy);
a complement of the first adaptor (comprising a complement of the original loop sequence (L′) flanked by complements of the base-paired stem of the first adaptor);
a copy of the 3′ end of the forward strand (of the original library fragment) (B copy);
a copy of the 5′ end of the forward strand (of the original library fragment) (A copy); and
a complement of a second strand of the first adaptor (comprising a complement of the second strand of the base-paired stem of the first adaptor and a complement of the primer-binding complement sequence (e.g. a first primer-binding sequence—e.g. P7′ for example, SEQ ID NO: 4 or a variant or fragment thereof)).

The reverse strand of the resulting amplified library strand will comprise (in the 3′ to 5′ direction);
a first strand of the second adaptor (comprising a second primer-binding sequence (e.g. P5′, for example, SEQ ID NO: 3 or 6 or a variant or fragment thereof) and a first strand of the base-paired stem);
the complement of the 5′ “half” of the original forward strand (i.e. the 3′ “half” of the reverse strand) (A′);
the complement of the 3′ “half” of the forward strand (i.e. the 5′ “half” of the reverse strand (B′);
the first adaptor, comprising a loop sequence (L) flanked by the base-paired stem of the first adaptor;
the 3′ “half” of the forward strand (B);
the 5′ “half” of the forward strand (A); and
a second strand of the first adaptor (comprising the second strand of the base-paired stem of the first adaptor and second primer-binding complement sequence (e.g. P7, for example, SEQ ID NO: 2 or a variant or fragment thereof)).

As shown in FIG. 4, although the amplified library strands are described to comprise a loop sequence (or loop complement sequence), this refers to the structure of the sequence when present in the first adaptor. The loop sequence in the amplified library strand may be a linear sequence. As such, this sequence may also be referred to as a linear first adaptor sequence (or just first adaptor sequence) or a loop sequence, and such terms may be used interchangeably herein, although when a “loop sequence” is used, for ease of reference, in the context of the amplified library strand it is not intended to limit its structure to a loop (i.e. a linear sequence is encompassed).

As also shown in FIG. 4, the orientation of the polynucleotide sequence (i.e. the insert) to be identified is reversed either side of the loop—i.e. the sequence is A-B-loop-B′-A′ (rather than A-B-loop-A′-B′, for example). This results in an inverted repeat tandem insert polynucleotide library strand. Such a polynucleotide may be referred to herein as an inverted-repeat tandem-insert polynucleotide library strand. As explained above, the expectation is that the complementary sequence of a double-stranded DNA molecule should contain the same (i.e. exactly complementary) information. This may not be the reality in practice for a number of reasons (for example DNA damage, e.g. oxidative damage to one or more bases of one strand). Sequencing an inverted-repeat tandem-insert polynucleotide library strand can be used to determine mismatches (e.g. asymmetry) between complementary strands.

Accordingly, in a further aspect of the invention, there is provided, as described further above, an inverted-repeat tandem-insert polynucleotide library strand, wherein the library strand comprises a primer-binding complement sequence, a first portion to be identified, a loop sequence, a second portion to be identified and a primer-binding sequence, wherein the first and second portions are complementary sequences and wherein the sequence of the second portion is inverted with respect to the first portion, and wherein the loop sequence comprises at least one restriction site for a nicking endonuclease. In a further embodiment, the primer-binding sequence and primer-binding complement sequence comprise at least one cleavable site and/or complement of a cleavable site. In one embodiment, the cleavable site is a restriction site. The inverted-repeat tandem-insert polynucleotide library strand may be single or double-stranded.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 or at least 50 base pairs and the second portion is at least 25 base pairs or at least 50 base pairs.

Sequencing of the termini of such inverted-repeat tandem-insert library strands results in equivalent sequences in the same direction (e.g. A-B-loop-B′-A′), whereby each end represents the sequence of a different strand of the original duplex (FIG. 4).

Where the library strand has not undergone modification, for example, an epigenetic conversion strategy has not been applied as described above the inverted-repeat tandem-insert library strand is susceptible to re-hybridisation during SBS. A solution to this problem is described below.

In one aspect of the invention, there is provided a method of identifying at least a first region of a polynucleotide sequence, wherein the method comprises
a. preparing at least one polynucleotide library strand as described above;
b. amplifying the polynucleotide library strand to generate a first and second library strand, wherein each library strand comprises a first and second region;
c. hybridising the first or second library strands to first and second immobilised primers respectively on a solid support and carrying out a first extension reaction to generate a first or second immobilised template strand;
d. hybridising the first or second immobilised template strands to a second or first immobilised primer respectively and carrying out a second extension reaction to generate a second and first immobilised template strand;
e. hybridising the first and second immobilised template strands;
f. applying a first endonuclease; and
g. sequencing the first and second immobilised template strands, wherein sequencing the first and second immobilised template strands identifies the first region.

In a further embodiment, the method comprises displacing or de-hybridising the (non-immobilised) library strands from the first or second immobilised strands and hybridising the first immobilised template strand to the 5′ end of the second immobilised strand (which comprises a 5′ primer sequence) or hybridising the second immobilised template strand to the 5′ end of the first immobilised strand (which also comprises a 5′ primer sequence). This allows extension of the second or first immobilised strands using the bridged first extension strand as a template. This step is referred to as clustering. In one embodiment, the cluster is generated by bridge amplification.

By “identification” or “identifying” is meant here obtaining genetic information from the polynucleotide strand or polynucleotide strands. This may include identification of the genetic sequence of the polynucleotide strand or polynucleotide strands (i.e. sequencing). Furthermore, this may instead, or additionally, include identification of mismatched base pairs. In addition, this may instead, or additionally, include identification of any epigenetic modifications, for example methylation. Accordingly, “identification” may mean identification of the genetic sequence of the polynucleotide strand or polynucleotide strands, mismatched base pairs, and/or identification of any epigenetic modifications.

In one embodiment, amplifying the polynucleotide library strand generates a first region to be identified and a second region (that may be also identified), such as on a single polynucleotide strand. As described above, the first and second regions may be complementary sequences, and are orientated as inverted-repeat tandem inserts—that is, both regions are on the same polynucleotide strand, and are inverted in sequence with respect to each other (as shown in FIG. 4). Accordingly, in one embodiment, the method comprises generating a plurality of inverted-repeat tandem-insert library strands, wherein each library strand comprises a first and second region. In one embodiment, the method further comprises de-hybridising the library strand to produce single-stranded inverted-repeat tandem-insert library strands.

In one embodiment, each first and second library strands comprises a primer-binding complement sequence, a first portion to be identified, a loop sequence, a second portion to be identified and a primer-binding sequence, wherein the first and second portions are complementary sequences and wherein the sequence of the second portion is inverted with respect to the first portion, and wherein the loop sequence comprises at least one restriction site (a first restriction site) for an endonuclease. In a further embodiment, the primer-binding sequence and primer-binding complement sequence comprise at least one cleavable site and/or at least one complement of a cleavable site. In one embodiment, the cleavable site/complement of cleavable site is a restriction site/complement of a restriction site.

The inverted-repeat tandem-insert polynucleotide library strand may be single or double-stranded.

In a further embodiment, the method comprises converting any epigenetic modifications (e.g. modified cytosines) using a conversion reagent, as described above.

In a further embodiment, the method comprises applying the plurality of inverted-repeat tandem-insert library strands in solution to a solid support (such as a flow cell), wherein, as described above, each inverted-repeat tandem-insert library strand comprises a first or second 3′ primer-binding sequence (e.g. P5′ or P7′), and wherein the solid support has immobilised thereon a plurality of lawn primer sequences complementary to the first and second 3′ primer-binding sequences.

In a further embodiment, the method comprises hybridising the 3′ primer binding sequence of the first library strand (a single stranded inverted-repeat tandem-insert library strand) to a first lawn primer or hybridising the 3′ primer binding sequence of the second library strand (a single stranded inverted-repeat tandem-insert library strand) to a second lawn primer; and carrying out an extension reaction to extend the lawn primers to generate a first or second immobilised (also referred to herein as extended) template strand complementary to the library strands, wherein the immobilised strands comprise a 3′ (second or first respectively) primer binding sequence. Accordingly, in one embodiment, the first and second library strands comprise a first and second 3′ primer-binding sequence, the solid support comprises a first and second immobilised primer, and the first and second library strands hybridise by their 3′ primer-binding sequences to the first and second immobilised primers.

In a further embodiment, the method comprises displacing or de-hybridising the (non-immobilised) library strands from the first or second immobilised strands and hybridising the first immobilised template strand to the 5′ end of the second immobilised strand (which comprises a 5′ primer sequence) or hybridising the second immobilised template strand to the 5′ end of the first immobilised strand (which also comprises a 5′ primer sequence). This allows extension of the second or first immobilised strands using the bridged first extension strand as a template. This step is referred to as clustering. In one embodiment, the cluster is generated by bridge amplification.

In a further embodiment, the method comprises hybridising the first immobilised template strand to the 5′ end of the second immobilised strand (which comprises a 5′ primer sequence) and hybridising the second immobilised template strand to the 5′ end of the first immobilised strand (which also comprises a 5′ primer sequence). This structure may be referred to herein as a sequence bridge. The sequence bridge is hybridised at a least three places: (1) the 5′ primer of the first extended strand is hybridised to the 3′ primer-binding region of the second extended strand (e.g. P5′); (2) the loop sequences of both the first and second extended strands and (3) the 5′ primer of the second extended strand (e.g. P7) is hybridised to 3′ primer-binding region of the first extended strand (e.g. P7′). Accordingly, this structure may be referred to herein as a loop-hybridised sequence bridge.

In a further embodiment, the method comprises applying (i.e. adding/flowing over the surface of the solid support), a first nicking enzyme. In one example, the nicking enzyme cleaves the first or second restriction sites within the template strand.

In one embodiment, the first nicking enzyme cleaves the first restriction sites. These are the restriction sites within the first adaptor (or present originally in the adaptor). In one embodiment, the first restriction site is in the loop sequence. In an alternative embodiment, the second restriction site is in the base-paired stem (that flank the loop sequence).

In another embodiment, the first nicking enzyme cleaves the second restriction sites. These are the restriction sites within the second adaptor. In one embodiment, the second restriction site is in base-paired stem (at the 3′ end of the second adaptor sequences in the single stranded template).

In one embodiment, following cleavage the sequences located 3′ of the cleaved sequence are de-hybridised and washed off.

In a further embodiment, the method comprises carrying out a first sequencing read to determine the sequence of the first and second immobilised strands simultaneously, such as by a sequencing-by-synthesis technique or by a sequencing—by ligation technique.

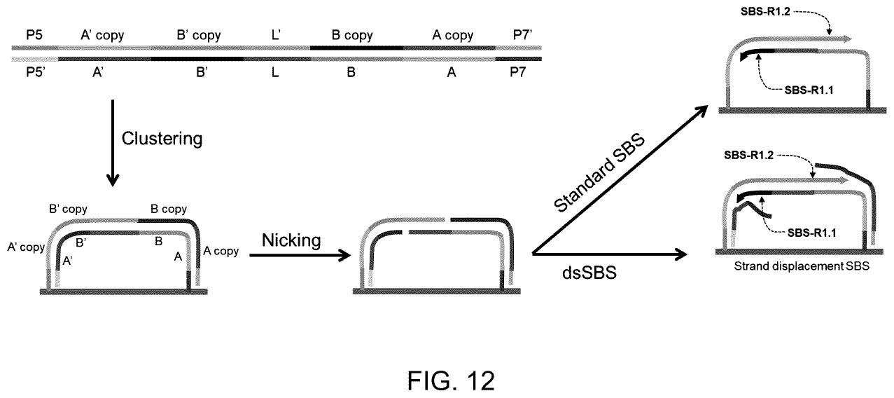

An example of a method of sequencing an inverted-repeat tandem-insert library strand is shown in FIG. 12. Each inverted-repeat tandem-insert duplex is de-hybridized, and the single strands flowed across a solid support (e.g. a flow cell) to attach to the solid support via Watson-Crick binding to a complementary lawn primer (P5 or P7) and become immobilised. The lawn primers (P5 and P7) are then extended (using the hybridised strand as a “template”) to generate a first or second immobilised template strand. For example, the first extended immobilised strand may comprise a first primer sequence at its' 5′ end (e.g. P5), and a first primer-binding sequence at its 3′ end (e.g. P7′). Similarly, the second extended immobilised strand may comprise a second primer sequence at its' 5′ end (e.g. P7), and a second primer-binding sequence at its 3′ end (e.g. P5′).

Following extension of the lawn primers to generate the first and second extended strands, the 3′ ends of each extended strand bend over to bind to the other, non-bound lawn adaptor (P7 or P5) to form a sequence bridge. As described above, this sequence bridge differs from conventional sequence bridges, as the sequence bridge is hybridised at at least three places-(1) the 5′ primer (e.g. P5) of the first extended strand is hybridised to the 3′ primer-binding region of the second extended strand (e.g. P5′); (2) the loop sequences of both the first and second extended strands and (3) the 5′ primer of the second extended strand (e.g. P7) is hybridised to 3′ primer-binding region of the first extended strand (e.g. P7′). As described above, this structure may be referred to herein as a loop-hybridised sequence bridge. The sequence bridge may be further hybridised within the regions to be identified.

In the next step, nicking enzymes are added. The nicking enzymes may be flowed across the solid support following clustering and formation of the loop-hybridised sequence bridge as described above.

As shown in FIG. 12, where the loop sequences (or loop complement sequences) comprise a 3′ restriction site (that is, the restriction site is at the 3′ end of the loop sequence) nicking enzymes may be applied to nick the sequence bridges at a pair of recognition sequences in the loop stem (e.g. the base-paired stem). This leaves the first extended strand and the second extended strand hybridised at the loop structure, each of which provide a sequencing start site for a different strand of the original duplex template. These strands can be simultaneously sequenced by standard SBS or double-stranded SBS (e.g. strand displacement SBS), as shown in FIG. 12. However, in all configurations of this workflow, the sequencing start sites are formed simultaneously by nicking enzymes, which therefore, allows both strands of the duplex to be sequenced simultaneously.

In standard SBS sequencing, the non-immobilised sequences—that is, the sequences 3′ of the nicked site—are washed off before addition of a read 1.1 (SBS-R1.2) and read 1.2 (SBS-R1.2) sequencing primer, which anneal to the nicked sites in the loop sequence of the first and second extended strands respectively, and a polymerase. As shown in FIG. 12, read 1.1 will sequence B′ and A′ (i.e. the reverse strand of the original duplex in the 3′ to 5′ direction) and read 1.2 will sequence B copy and A copy (the copy of the forward strand of the original duplex in the 3′ to 5′ direction). This allows for any errors in the reverse strand to be identified.

In double-stranded SBS (e.g. strand displacement SBS), the non-immobilised sequences 3′ of the nicked site are not washed off.

Single-strand displacement SBS is an effective method for the sequencing of the prepared duplex. This method requires a nick in the duplex sequence and primers for DNA polymerase to utilise, to incorporate reversibly-terminated labelled dNTPs into a complementary strand of one strand of the template.

Single-strand displacement SBS combines the principles of single strand replication and sequencing-by-synthesis technologies to sequence duplexes. In single-strand displacement SBS, a DNA polymerase capable of strand-displacement but lacking exonuclease activity, such as phi29 DNA polymerase, is utilised. DNA polymerases lacking exonuclease activity in both the 5′-3′ and 3′-5′ direction are required, to allow for both Reads 1 and 2. The nick site within the duplex target and annealed primer provides a binding site for such a DNA polymerase to bind. After docking, the DNA polymerase extends the primer adjacent to the nick site to generate a sequencing strand. The sequencing strand is formed by incorporating labelled deoxynucleoside triphosphates (dNTP), complementary to the relevant template strand. The labelled dNTPs act as a terminator for polymerization, so after each dNTP incorporation, the fluorescent dye is imaged to identify the base and then enzymatically cleaved to allow incorporation of the next nucleotide. Since all four reversible terminator-bound dNTPs (A, C, T, G) are present as single, separate molecules, natural competition minimizes incorporation bias. Simultaneous to polymerising a complementary strand, the DNA polymerase uses its strand displacement activity to displace the other “non-template” strand for access. In this invention, this workflow occurs simultaneously for each read (R1.1 and R1.2/R2.1 and R2.2).

FIG. 6 describes an alternative method of sequencing an inverted-repeat tandem-insert template. A sequence bridge is formed as described in FIG. 3. In this example, the 3′ end of the lawn primer sequences (e.g. both P5 and P7) comprise a restriction site (the second restriction site) as described above. This restriction site is the complement of the restriction site present in the base-paired stem of the second adaptor. Simultaneous nicking of these restriction sites provides two sequencing start sites that allow simultaneous sequencing from the opposite end of both inserts, i.e. 5′ to 3′ direction—and at opposite ends of the insert to FIG. 12. As described in FIG. 6, these strands can be simultaneously sequenced by double-stranded SBS, such as strand displacement SBS. As shown in FIG. 6, read 1.1 (SBS R1.1) will sequence A′ copy and B′ copy (the copy of the reverse strand of the original duplex in the 5′ to 3′ direction) and read 1.2 (SBS R1.2) will sequence A and B (the forward strand of the original duplex in the 5′ to 3′ direction). This allows for any errors in the forward strand to be identified.

As shown in FIG. 7, a 9QAM encoding scheme can be used to accurately differentiate between two simultaneously received base calls. By plotting relative intensities of light signals obtained from Read 1.1 and Read 1.2 a constellation of 9 clouds is obtained. Each of these clouds allows sequence information to be identified from the two reads; in this particular encoding scheme, the top left corner of four clouds corresponds with base calls corresponding to A, the top right corner of four clouds corresponds with base calls corresponding to T, the bottom left corner of four clouds corresponds with base calls corresponding to G, and the bottom right corner of four clouds corresponds with base calls corresponding to C; however, other encoding schemes are possible and each of C, G, A and T may be mapped to different cloud permutations. By plotting the light intensities in this manner it is possible to determine an accurate base call from a library prep or sequencing error (and by library prep or sequencing error is meant here that there is a mismatch between read 1.1 and read 1.2, which may be indicative of asymmetry between the forward and reverse strands, for example, because of DNA damage to one strand).

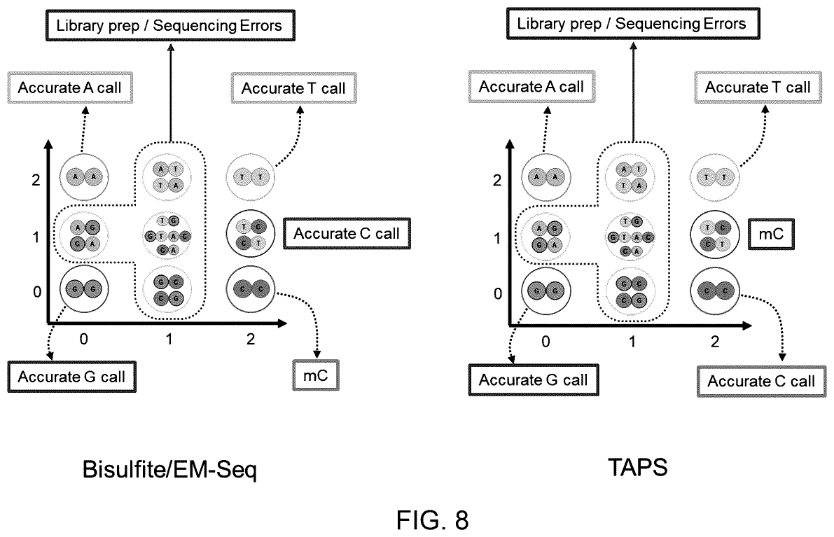

The method described herein can also be used to simultaneously sequence genomic and epigenetic data. Following preparation of the polynucleotide library strand, an epigenetic conversion is applied. The modified library strand can then be sequenced as described above and the sequences of the duplex strands read simultaneously. A 9 QaM system is used to decode the simultaneously-received read signals. Depending on which technology for epigenetic conversion is used, the C/C cloud may either represent a mC (Bisulfite/EM-Seq) or accurate C call (TAPS) and vice versa, the C/T cloud will represent the mC or accurate C calls respectively (FIG. 8).

Following sequencing of one strand of the duplex (i.e. read 1) as described above, sequencing of the other, second strand of the duplex can be carried out using either single stranded or double stranded SBS.

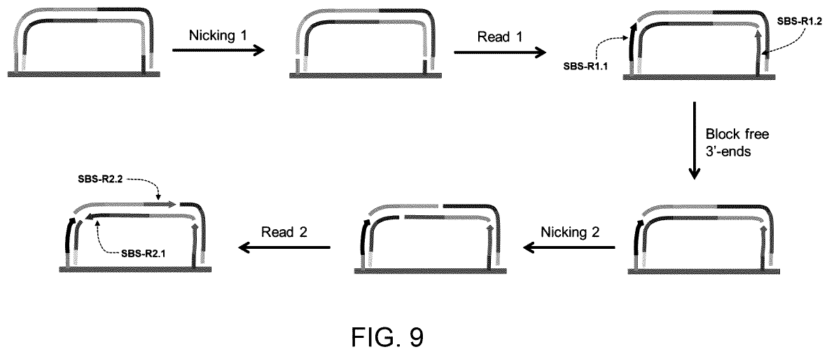

In one example, as shown in FIG. 9, following nicking of the lawn primers (as shown in FIG. 6 or 12) and sequencing of the first strand (read 1), the free ends of the sequenced strands are blocked. By “free ends” is meant the free 3′ hydroxyl group of the 3′ end or 3′ nucleotide of an extended polynucleotide strand.

Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the lawn primer), a hydrogen atom instead of a 3′-OH group, a phosphate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl (dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the free end by a polymerase. Alternatively, instead of blocking the free ends, these strands are extended to regenerate the polynucleotide strand (i.e. to resynthesized to generate the 3′ primer-binding sequences).

In the next step, nicking enzymes may be applied to nick the sequence bridges at the restriction sites within the loop sequence (or loop complement sequence), using an alternative recognition site to the first nicking event. That is, nicking occurs at the restriction sites at the 3′ end of the loop sequence. As shown in FIG. 9, this generates two start sites for sequencing allowing simultaneous sequencing of the other strand of the original polynucleotide duplex. For example, as shown in FIG. 9, read 2.1 (SBS-R2.1) will sequence B′ and A′ (i.e. the reverse strand of the original duplex in the 3′ to 5′ direction) and read 2.2 (SBS-R2.2) will sequence B copy and A copy (the copy of the forward strand of the original duplex in the 3′ to 5′ direction). This allows for any errors in the reverse strand to be identified. In this example, read 2 may be sequenced by either single or double-stranded SBS, as described above.

The two reads, each with simultaneous sequencing of two strands—as described for example in FIGS. 6 and 9—allows the entire inverted-repeat tandem-insert duplex to be sequenced.

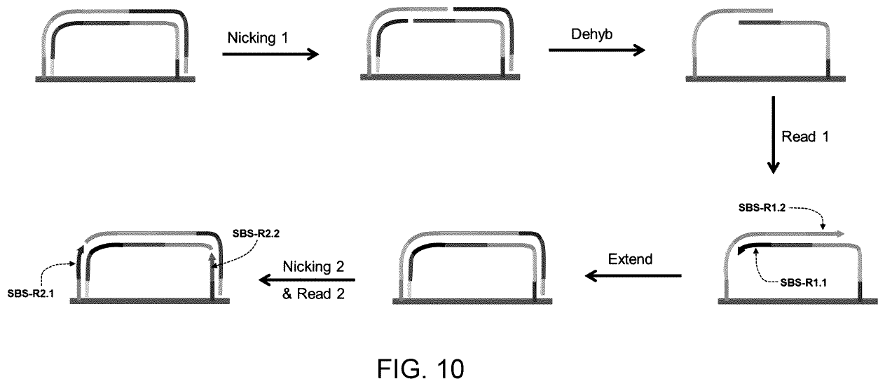

The order of nicking reactions can also be reversed. For example, the first nicking step may be nicking of the loop sequence and the second nicking step may be nicking of the 3′ end of the primer sequence. This is shown, for example in FIG. 10.

As shown in FIG. 10, read 1 is generated following the method described in FIG. 12. This allows for any errors in the forward strand to be identified. Sequencing may be single-stranded or double-stranded SBS.

The sequenced strands are then extended (i.e. resynthesized) to regenerate the 3′ primer-binding sequences. In the next step, nicking enzymes may be applied to nick the sequence bridges at the 3′ end of the primer sequences (as described, for example, in FIG. 10). Simultaneous nicking of these restriction sites provides two sequencing start sites that allow simultaneous sequencing from the opposite end of both inserts, i.e. 5′ to 3′ direction—and at opposite ends of the insert to FIG. 12. As described in FIG. 10, these strands can be simultaneously sequenced by double-stranded SBS, such as strand displacement SBS. As shown in FIG. 10, read 2.1 (SBS R2.1) will sequence A′ copy and B′ copy (the copy of the reverse strand of the original duplex in the 5′ to 3′ direction) and read 2.2 (SBS R2.2) will sequence A and B (the forward strand of the original duplex in the 5′ to 3′ direction). This allows for any errors in the forward strand to be identified.

Accordingly, in a further embodiment, following read 1, the method comprises blocking all or substantially all free 3′ ends of the immobilised strands. Alternatively, following read 1, each immobilised strand is extended to regenerate the loop-hybridised sequence bridge described (as shown in FIG. 10). Therefore, in one embodiment, the method comprises carrying out an extension reaction to extend each immobilised strand.

In a further embodiment, the method further comprises applying (i.e. adding/flowing over the surface of the solid support), a second nicking enzyme. In one embodiment, the second nicking enzyme cleaves the first or second restriction sites within the template strand. In another embodiment, the second nicking enzyme cleaves a different restriction site from the first nicking enzyme. Accordingly, where the first nicking enzyme cleaves the first restriction site, the second nicking enzyme cleaves the second restriction site (as shown in FIG. 10). Similarly, where the first nicking enzyme cleaves the second restriction site, the second nicking enzyme cleaves the first restriction site (as shown in FIG. 9).

In one embodiment, following read 1, and where the first nicking enzyme has cleaved the second restriction site, the method comprises blocking all or substantially all free 3′ ends of the immobilised strands, and applying a second nicking enzyme where the second nicking enzyme cleaves the first restriction site (as shown in FIG. 9).

In an alternative embodiment, following read 1, and where the first nicking enzyme has cleaved the first restriction site, the method comprises carrying out an extension reaction to extend the immobilised strands, and applying a second nicking enzyme where the second nicking enzyme cleaves the second restriction site as shown in FIG. 10).

In a further embodiment, the method comprises carrying out a second sequencing read to determine the sequence of the first and second immobilised strands simultaneously, such as by a sequencing-by-synthesis technique or by a sequencing—by ligation technique. This sequence read is read 2.

In an alternative embodiment, the method comprises generating a sequence bridge, as described above, and simultaneously cleaving both strands of the bridge. This is possible if the first restriction site is in the middle of the loop or substantially the middle of the loop.

In one embodiment, the endonuclease is a double strand restriction endonuclease or restriction enzyme. By either of these terms is meant an enzyme that can hydrolyze both strands of the double-stranded polynucleotide (duplex), to produce DNA molecules that are cleaved on both strands. In one embodiment, the restriction enzyme is a type II restriction enzyme. In one example, the type II restriction enzyme is EcoRI and the restriction enzyme is G/AATTC wherein EcoRI catalyzes a double stranded break within the recognition site. In another example, the type II restriction enzyme is Bg1II and the restriction site is A/GATCT, wherein Bg1II catalyzes a double stranded break within the recognition site. In a further example, the type II restriction enzyme is Notl and the restriction site is GC/GGCCGC, wherein Notl catalyses a double stranded break within the recognition site.

Furthermore, in this embodiment, the loop sequence in the first adaptor will comprise the following structure: first sequencing primer-binding sequence-restriction site-complement of a second sequencing primer-binding sequence. As a result, the first immobilised template (within the loop sequence) will comprise a first sequencing primer-binding sequence, a restriction site and a complement of a second sequencing primer-binding sequence, and the second immobilised template will comprise a complement of a first sequencing primer-binding sequence, a restriction site and a complement of a second sequencing primer-binding sequence. The first and second sequencing primer-binding sequences bind a sequencing primer, which may be the same sequence. That is, they bind the same sequencing primer. Alternatively, the first and second sequencing primer-binding sequences are different. That is, they bind different sequencing primers.

The sequencing primer-binding sequences may be in the base-paired stem of the loop sequence.

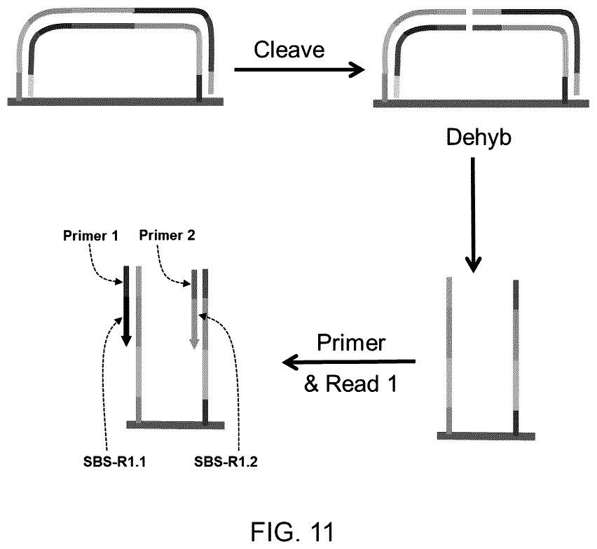

Following nicking of the loop sequence two immobilised extended strands are generated—a first immobilised extended strand and a second immobilised extended strand, as shown in FIG. 11. In effect, this step halves the tandem insert. Each immobilised extended strand has a 3′ sequencing primer-binding sequence (either a first sequencing primer-binding sequence or a second sequencing primer-binding sequence). Non-immobilised strands may be washed off.

Binding of a first sequencing primer to the first sequencing primer-binding sequence will allow sequencing of read 1.1. As shown in FIG. 11.

Binding of a second sequencing primer to the second sequencing primer-binding sequence will allow sequencing of read 1.2. As shown in FIG. 11.

In one embodiment, binding of first sequencing primers to the first sequencing primer-binding sequence generates a first signal and binding of second sequencing primers to the second sequencing primer-binding sequence generates a second signal, where the intensity of the first signal is greater than the intensity of the second signal. This allows read 1.1 and 1.2 to be read simultaneously. This is achieved using a mixed population of blocked and unblocked second sequencing primers that bind the second sequencing primer-binding site. Any ratio of blocked:unblocked second primers can be used that generates a second signal that is of a lower intensity than the first signal, for example, the ratio of blocked:unblocked primers may be: 20:80 to 80:20, or 1:2 to 2:1. In one embodiment, a ratio of 50:50 of blocked:unblocked second primers is used, which in turn generates a second signal that is around 50% of the intensity of the first signal.

The first and second sequencing primers may be added to the flow cell at the same time, or separately but sequentially.

By “blocked” is meant that the sequencing primer comprises a blocking group at a 3′ end of the sequencing primer. Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

The sequence of the sequencing primers and the sequence primer binding sites are not material to the methods of the invention, as long as the sequencing primers are able to bind to the sequence primer-binding site to enable amplification and sequencing of the regions to be identified.

In summary, the above-described example would allow spatially separated clusters to be read in a temporally simultaneous manner through the generation of an optically unresolved signal that can be analytically separated using 16 QaM.

In a further embodiment, the method may additionally comprise generating a complement of the read 1 sequences (i.e. a complement of the halves the tandem insert shown in FIG. 10), and sequencing the complements as described above (i.e. following the same method of FIG. 10 with sequencing primers that bind complements of the first and second primer-binding sequences). This allows sequencing of read 2. Again, binding of first sequencing primers to the complement of the first sequencing primer-binding sequence generates a first signal and binding of second sequencing primers to the complement of the second sequencing primer-binding sequence generates a second signal, where the intensity of the first signal is greater than the intensity of the second signal allows read 2.1 and 2.2 to be read simultaneously. In one embodiment, the complements of the read 1 sequences may be obtained by modifying the solid support such that the solid support additionally comprises lawn primers (third and fourth lawn primers) that are complementary to the or at least a portion of the first and second primer-binding sequences. Binding of the 3′ end of the immobilised read 1 sequences (e.g. last diagram of FIG. 11) to third and fourth primers (not shown) leads to formation of a bridge. The third and fourth lawn primers can be extended using bridge amplification and sequenced using the methods described above.

Accordingly, in an alternative embodiment, the method of identifying a polynucleotide, comprises applying (i.e. adding/flowing over the surface of the solid support), a first restriction enzyme, wherein the restriction enzyme cleaves the first restriction site, wherein the first restriction site is in the loop sequence of the first adaptor. In one embodiment, following cleavage the sequences 3′ of the cleaved sequence are de-hybridised and washed off.

In a further embodiment, the method comprises carrying out a first sequencing read to determine the sequence of the first and second immobilised strands simultaneously, such as by a sequencing-by-synthesis technique or by a sequencing—by ligation technique.

### Kits

In another aspect of the invention, there is provided a library preparation kit, wherein the kit comprises a plurality of first adaptors, a plurality of second adaptors. In one embodiment, the kits further comprises instructions for use. In a further embodiment, the kit may further comprise at least one single-stranded endonuclease or restriction endonuclease. In one aspect, the endonuclease is selected from Nt. BspQI, Cas9 D10A and Cas9 H840A.

In another embodiment, the kit may additionally comprise an agent for epigenetic conversion. For example, the agent for epigenetic conversion may be a conversion agent as described herein Non-limiting examples of conversion reagents include sulfites (e.g. bisulfite), cytidine deaminases (e.g. wild-type or mutant enzymes of the APOBEC family), and boron-based reducing agents (e.g. amine-borane compounds or azine-borane compounds, such as t-butylamine borane, ammonia borane, ethylenediamine borane, dimethylamine borane, pyridine borane and 2-picoline borane).

In another embodiment the kit may additionally comprise a uracil glycosylase or USER enzyme mix (which is a cocktail of uracil glycosylase and endonuclease VIII).

In another aspect of the invention there is provided a solid support comprising a plurality of a third and/or fourth primer immobilised thereon, as described above.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. The term “partially” is used to indicate that an effect is only in part or to a limited extent.

Unless otherwise explicitly stated, articles such as “a” or “an” should generally be interpreted to include one or more described items.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The present invention will now be described by way of the following non-limiting examples.

### EXAMPLES

### Example 1—Mismatched Base Pair Analysis on NA12878 Sample Using 9 QaM

### Oligo Sequences:

Asterisk (*) indicates a phosphorothioate linkage.

Bold indicates nicking restriction site (or its complement) of Nt.BspQI, which recognises the following sequence (nicking site is indicated by arrow):

5′...G C T C T T C N^{▾}...3′
3′...C G A G A A G N...5′

[Biotin-T] indicates the following structure:

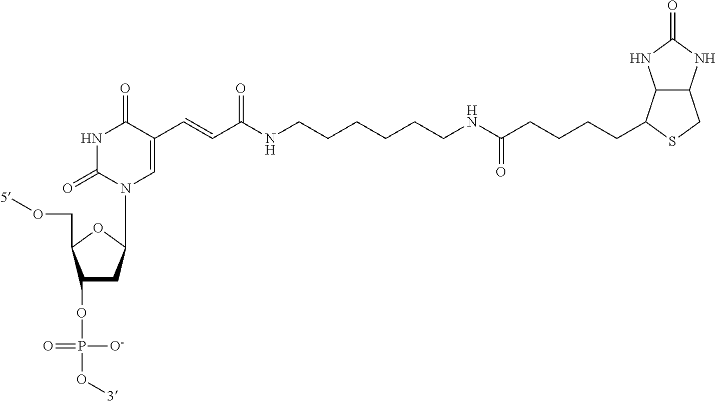

P5_BbvCI_P7 (SEQ ID NO: 7):
GCTGAGGATCTCGTATGCCGTCTTCTGCTTGUAATGATACGGCGACCACC
GAGATCTACACTCCTCAGC*T
BspQI_iSce_Loop (SEQ ID NO: 8):
GAAGAGCACACGTCTGAACTCCAGTCACTAGGGA[ Biotin-T]AACAG
GGTAATCTTTCCCTACACGACGCTCTTC*T

### Adaptor Annealing:

1. A mixture of 4 μl of 100 μM P5_BbvCI_P7 oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed P5_BbvCI_P7 adaptor.
2. Separately, a mixture of 4 μl of 100M BspQI_iSce_Loop oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed BspQI_iSce_Loop adaptor.
3. Equal volumes of the 20 μM stock of annealed P5_BbvCI_P7 adaptor from step 1 and 20 μM stock of annealed BspQI_iSce_Loop adaptor from step 2 are mixed together, giving a stock solution with 10 μM each of annealed P5_BbvCI_P7 adaptor and annealed BspQI_iSce_Loop adaptor.

### Preparation of Library:

1. NEB Ultra II FS reagents were thawed at room temperature and kept on ice until use.
2. The Ultra II FS Enzyme mix was vortexed for 5-8 seconds prior to use and placed on ice.
3. In a 0.2 ml PCR tube on ice, 26 μl DNA (100 ng of input DNA (NA12878 sample) diluted to 26 μl with Milli-Q grade water), 7 μl of NEBNext Ultra II FS Reaction Buffer and 2 μl of NEBNext Ultra II FS Enzyme Mix were added, briefly vortexed and spun in a microcentrifuge to mix.
4. In a Thermocycler with the heated lid set to 75 C, the tubes were incubated for 5 mins at 37 C, then 30 mins at 65 C then held at 4 C.
5. The following were added to the FS reaction mixture from step 4: 30 μl of NEBNext Ultra II Ligation Master Mix, 1 μl of NEBNext Ligation Enhancer and 2.5 μl of the loop adaptors P5_BbvCI_P7 and BspQI_iSce_Loop (10 μM each) prepared from step 3 of “Adaptor annealing”.
6. The entire volume was pipetted up and down 10× to mix, followed by a brief spin in a microcentrifuge.
7. The mixture was incubated at 20 C for 15 mins in a thermocycler with the heated lid off.
8. 3 μl of USER Enzyme (NEB) was added to the ligation mix.
9. The mixture was mixed well and incubated at 37 C for 15 mins with the heated lid set to >47 C.
10. Adaptor ligated DNA was then size selected via a 0.8× SPRI (iTune beads) selection: 40 μl iTune beads (ILMN) were added to 68.5 μl of ligation reaction, mixed and incubated at RT for 5 mins.
11. The mixture was placed on a magnet for 5 mins, and the supernatant was discarded.
12. The beads were washed twice with 200 μl of 80% ethanol-200 μl 80% ethanol was added with beads on the magnet, followed by a 30 s wait, and ethanol was removed, then the wash was repeated once more.
13. The last remnants of ethanol were removed with a P10 pipette and tip.
14. Beads were then air dried for 5 mins.
15. DNA was eluted from beads with 40 μl of 0.1× TE buffer.
16. A second size selection was conducted via another 0.8× SPRI (iTune beads) selection: 20 μl iTune beads (ILMN) were added to 68.5 μl of ligation reaction, mixed and incubated at RT for 5 mins.
17. The mixture was placed on a magnet for 5 mins, and the supernatant was discarded.
18. The beads were washed twice with 200 μl of 80% ethanol-200 μl 80% ethanol was added with beads on the magnet, followed by a 30 s wait, and ethanol was removed, then the wash was repeated once more.
19. The last remnants of ethanol were removed with a P10 pipette and tip.
20. Beads were then air dried for 5 mins.
21. DNA was eluted from beads with 15 μl of 0.1× TE buffer, of which 7.5 μl was taken forward to the next step.
22. 175 μl of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).
23. The tubes were placed on a magnet until the beads pelleted.
24. The beads were washed twice with 200 μl of Tagmentation Wash Buffer (TWB, Illumina).
25. The beads were then washed once with 200 μl of Resuspension Buffer (RSB, Illumina).
26. The beads were resuspended in 20 μl of Milli-Q grade water and transferred to 0.2 ml tubes for the final PCR.
27. 20 μl of beads+DNA were combined with 25 μl of Illumina Enhanced PCR Mix (EPM) and 5 μl of PPC (PCR Primer Cocktail, Illumina).
28. The mixture was amplified by PCR: cycling procedure-98 C for 3 min followed by 12 cycles of (98 C 45 s, 60 C 2 min, 68 C 2 min), then 68 C for 5 mins and then hold at 4 C.
29. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq.
1. 400 μl BspQI mix was made up-360 μl Milli-Q grade water, 40 μl of rNEB3.1 buffer (NEB) and 8 μl of Nt.BspQI (NEB were combined). The mixture was vortexed to mix and briefly spun down. The mixture was pipetted into the “EXT” position of the MiniSeq cartridge (position to the left of the Custom Primer positions).
2. The library was denatured (0.1N NaOH) and diluted to 0.5 pM final concentration in HT1 buffer according to Illumina protocol. 500 μl was loaded into the “Library” position of the MiniSeq cartridge.
3. Setup was run using MiniSeq Control Software, using a standard MiniSeq run.

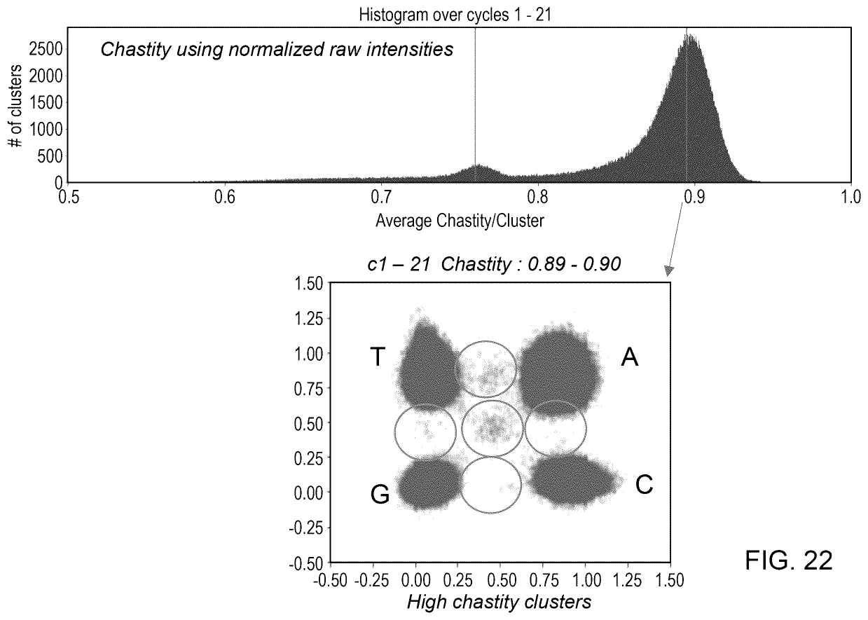

The 9 QaM results are shown in FIG. 22, where mismatched base pairs can be identified by analysing base calls that appear in the side or central clouds, rather than the four corner clouds. The centre middle cloud is one of the more populated clouds corresponding to mismatched base pairs, and this can primarily be attributed to (oxo-G)-A mismatched base pairs.

Overall, these results show that analysis can be conducted on polynucleotide sequences to identify mismatched base pairs. In particular, by enabling concurrent sequencing of the forward and reverse complement strands of the template (or reverse and forward complement strands of the template), mismatched base pairs can be identified quickly and accurately. Such a process is made viable by using the methods of preparing polynucleotide libraries as described herein.

### Example 2—Methylation Analysis on Methylated pUC19 Sample Using 9 QaM

### Oligo Sequences:

Asterisk (*) indicates a phosphorothioate linkage.

Underline indicates 5-methylcytosine instead of cytosine (in “P5_BbvCI_P7-methylated” and “BspQI_iSce_Loop-methylated”, all cytosines are replaced with 5-methylcytosines to prevent unwanted conversion of cytosine to uracil in the adaptor sequence during bisulfite conversion).

Bold indicates nicking restriction site (or its complement) of Nt.BspQI, which recognises the following sequence (nicking site is indicated by arrow):

5′...G C T C T T C N^{▾}...3′
3′...C G A G A A G N...5′

[Biotin-T] indicates the following structure:

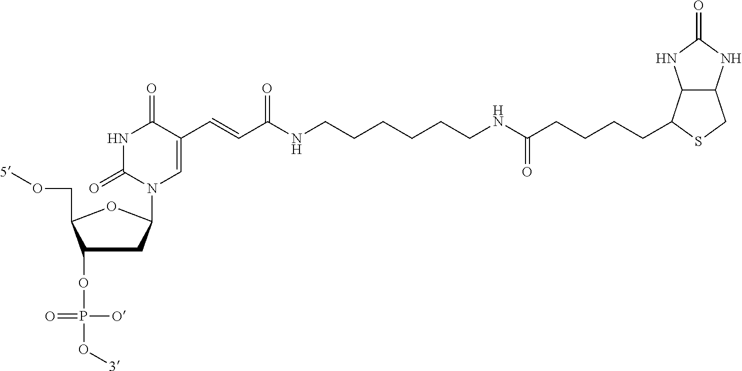

P5_BbvCI_P7 (SEQ ID NO: 7):
GCTGAGGATCTCGTATGCCGTCTTCTGCTTGUAATGATACGGCGACCACC
GAGATCTACACTCCTCAGC*T
BspQI_iSce_Loop (SEQ ID NO: 8):
GAAGAGCACACGTCTGAACTCCAGTCACTAGGGA[Biotin-T]AACAGG
GTAATCTTTCCCTACACGACGCTCTTC*T
P5_BbvCI_P7-methylated (SEQ ID NO: 9):
GCTGAGGATCTCGTATGCCGTCTTCTGCTTGUAATGATACGGCGACCACC
GAGATCTACACTCCTCAGC*T
BspQI_iSce_Loop-methylated (SEQ ID NO: 10):
GAAGAGCACACGTCTGAACTCCAGTCACTAGGGA[Biotin-T]AACAGG
GTAATCTTTCCCTACACGACGCTCTTC*T

### Adaptor Annealing:

1. A mixture of 4 μl of 100 μM P5_BbvCI_P7-methylated oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed P5_BbvCI_P7-methylated adaptor.
2. Separately, a mixture of 4 μl of 100 μM BspQI_iSce_Loop-methylated oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed BspQI_iSce_Loop-methylated adaptor.
3. Equal volumes of the 20 μM stock of annealed P5_BbvCI_P7-methylated adaptor from step 1 and 20 μM stock of annealed BspQI_iSce_Loop-methylated adaptor from step 2 are mixed together, giving a stock solution with 10 μM each of annealed P5_BbvCI_P7-methylated adaptor and annealed BspQI_iSce_Loop-methylated adaptor.

### Preparation of Library:

1. NEB Ultra II FS reagents were thawed at room temperature and kept on ice until use.
2. The Ultra II FS Enzyme mix was vortexed for 5-8 seconds prior to use and placed on ice.
3. In a 0.2 ml PCR tube on ice, 26 μl DNA (100 ng of input DNA (methylated pUC19 sample) diluted to 26 μl with Milli-Q grade water), 7 μl of NEBNext Ultra II FS Reaction Buffer and 2 μl of NEBNext Ultra II FS Enzyme Mix were added, briefly vortexed and spun in a microcentrifuge to mix.
4. In a Thermocycler with the heated lid set to 75 C, the tubes were incubated for 5 mins at 37 C, then 30 mins at 65 C then held at 4 C.
5. The following were added to the FS reaction mixture from step 4: 30 μl of NEBNext Ultra II Ligation Master Mix, 1 μl of NEBNext Ligation Enhancer and 2.5 μl of the loop adaptors P5_BbvCI_P7-methylated and BspQI_iSce_Loop-methylated (10 μM each) prepared from step 3 of “Adaptor annealing”.
6. The entire volume was pipetted up and down 10× to mix, followed by a brief spin in a microcentrifuge.
7. The mixture was incubated at 20 C for 15 mins in a thermocycler with the heated lid off.
8. 3 μl of USER Enzyme (NEB) was added to the ligation mix.
9. The mixture was mixed well and incubated at 37 C for 15 mins with the heated lid set to >47 C.
10. Adaptor ligated DNA was then size selected via a 0.8× SPRI (iTune beads) selection: 57 μl iTune beads (ILMN) were added to 68.5 μl of ligation reaction, mixed and incubated at RT for 5 mins.
11. The mixture was placed on a magnet for 5 mins, and the supernatant was discarded.
12. The beads were washed twice with 200 μl of 80% ethanol-200 μl 80% ethanol was added with beads on the magnet, followed by a 30 s wait, and ethanol was removed, then the wash was repeated once more.
13. The last remnants of ethanol were removed with a P10 pipette and tip.
14. Beads were then air dried for 5 mins.
15. DNA was eluted from beads with 40 μl of 0.1× TE buffer. At this stage, 20 μl was saved as a “non-converted” control, the remaining 20 μl was treated to bisulfite conversion, following the Zymo Research EZ-96 DNA Methylation Gold MagPrep kit (steps 16-25 are taken from the instructions for this kit).
16. In a 0.2 ml PCR tube, 20 μl of 0.8× SPRI selected ligation and 130 μl of CT Conversion Reagent (comprises sodium metabisulfite) were added.
17. The mixture was incubated on a thermocycler at 98 C for 10 mins, then 64 C for 2.5 hours, followed by holding at 4 C for up to 20 hours.
18. The sample was transferred to 1.7 ml tubes for subsequent steps. 600 μl of M-Binding Buffer and 10 μl of MagBinding Beads were added. The mixture was vortexed for 30 s.
19. Incubate at RT for 5 mins, then place on a magnet for 5 mins.
20. The supernatant was removed and discarded. 400 μl of M-Wash buffer was added to the beads, and then vortexed for 30 s. The mixture was placed back on magnet until the beads pelleted.
21. The supernatant was removed and discarded.
22. 200 μl of M-Desulphonation Buffer was added to the beads, and then vortexed for 30 s. The mixture was incubated at RT for 15-20 mins. The mixture was then placed back on magnet until beads pelleted.
23. The supernatant was removed and discarded. 400 μl of M-Wash buffer was added to the beads, then vortexed for 30 s. The mixture was placed back on magnet until beads pelleted. This wash step was repeated once.
24. The supernatant after 2nd wash was removed, and the tubes were transferred to a hot block at 55 C to air dry the beads for 20-30 mins and remove residual M-Wash buffer.
25. 25 μl of M-Elution Buffer was added to the dried beads and vortexed for 30 s. The elution mixture was heated at 55 C for 4 mins then the tubes were placed back on the magnet for 1 min (or until the beads pelleted). The eluate was removed and transferred to a new 1.7 ml tube.
26. 175 μl of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).
27. The tubes were placed on a magnet until the beads pelleted.
28. The beads were washed twice with 200 μl of Tagmentation Wash Buffer (TWB, Illumina).
29. The beads were then washed once with 200 μl of Resuspension Buffer (RSB, Illumina).
30. The beads were resuspended in 20 μl of Milli-Q grade water and transferred to 0.2 ml tubes for the final PCR.
31. 20 μl of beads+DNA were combined with 25 μl of Q5U Mastermix (NEB) and 5 μl of PPC (PCR Primer Cocktail, Illumina).
32. The mixture was amplified by PCR: cycling procedure-98 C for 3 min followed by 12 cycles of (98 C 45 s, 60 C 2 min, 68 C 2 min), then 68 C for 5 mins and then hold at 4 C.
33. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq.
1. 400 μl BspQI mix was made up-360 μl Milli-Q grade water, 40 μl of rNEB3.1 buffer (NEB) and 8 μl of Nt.BspQI (NEB were combined). The mixture was vortexed to mix and briefly spun down. The mixture was pipetted into the “EXT” position of the MiniSeq cartridge (position to the left of the Custom Primer positions).
2. The library was denatured (0.1N NaOH) and diluted to 0.5 pM final concentration in HT1 buffer according to Illumina protocol. 500 μl was loaded into the “Library” position of the MiniSeq cartridge.
3. Setup was run using MiniSeq Control Software, using a standard MiniSeq run.
4. For a CA dye swap, standard IMX was removed from the IMX position of the MiniSeq cartridge, then the position was washed 5 times with Milli-Q grade water, and replaced with 20 mls of custom IMX, where the standard two-dye system for A (A represented by red and green) and one-dye system for C (C represented by red) is replaced with a two-dye system for C (C represented by red and green) and one-dye system for A (A represented by red).

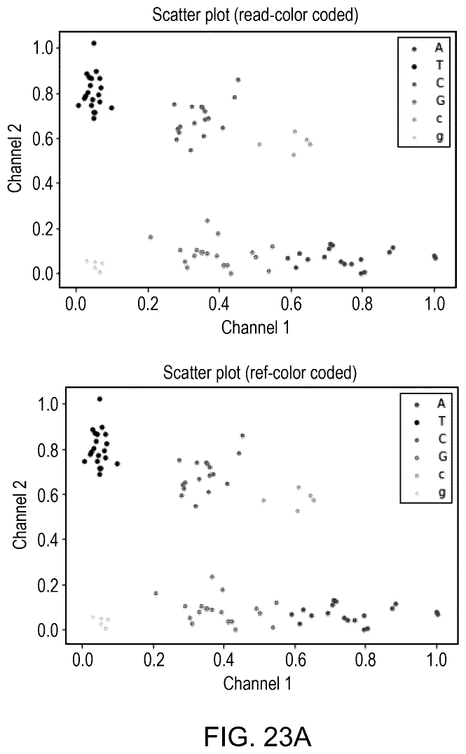

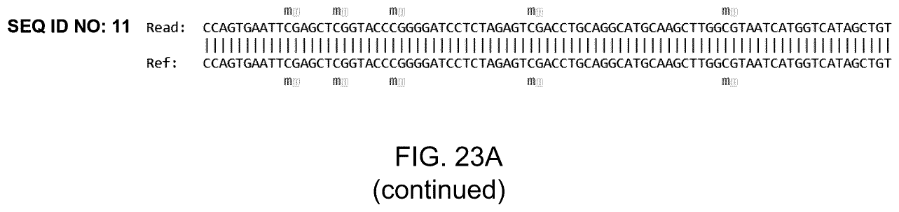

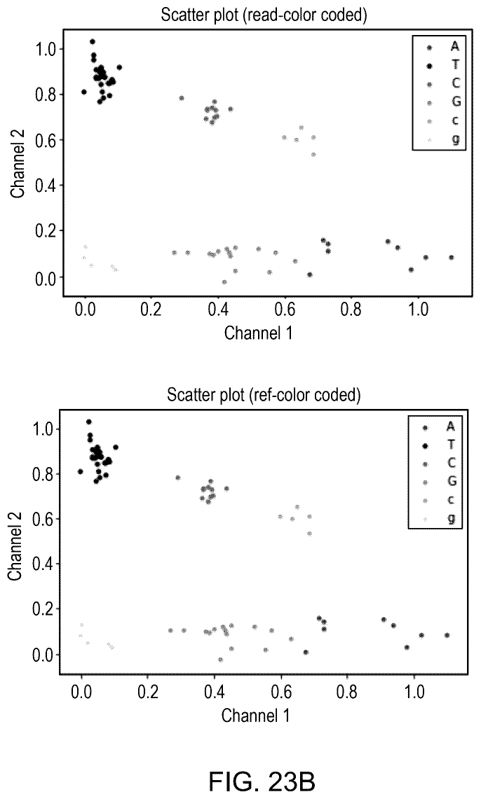

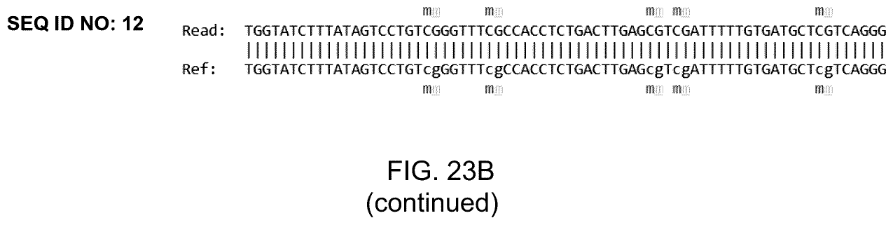

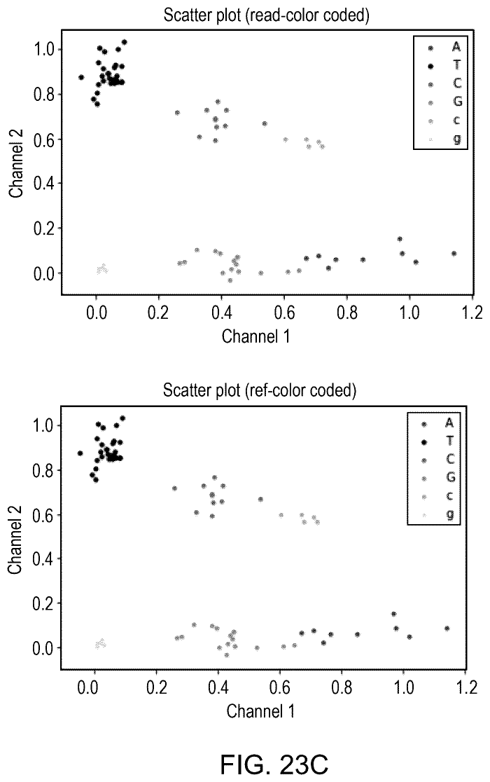

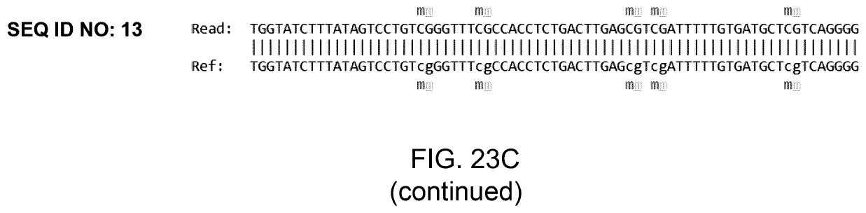

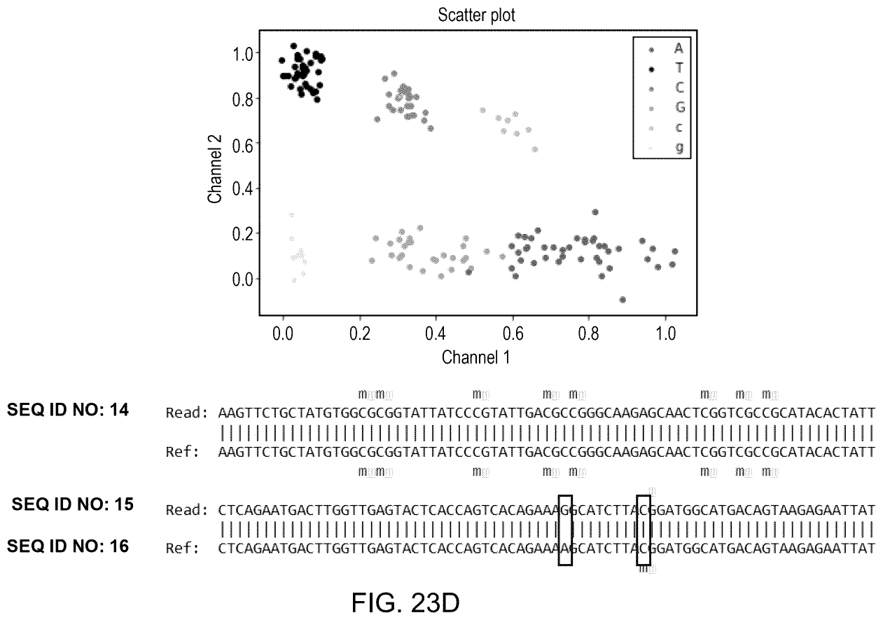

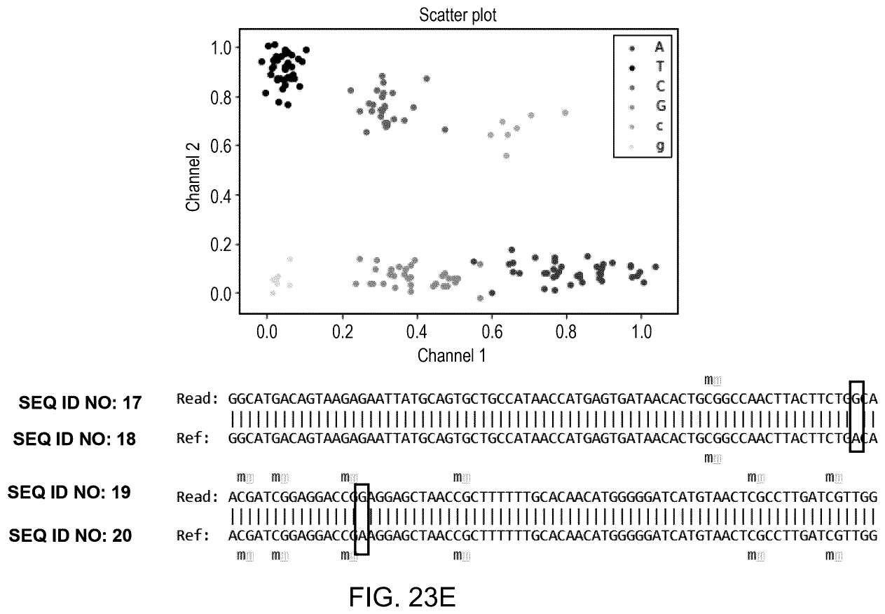

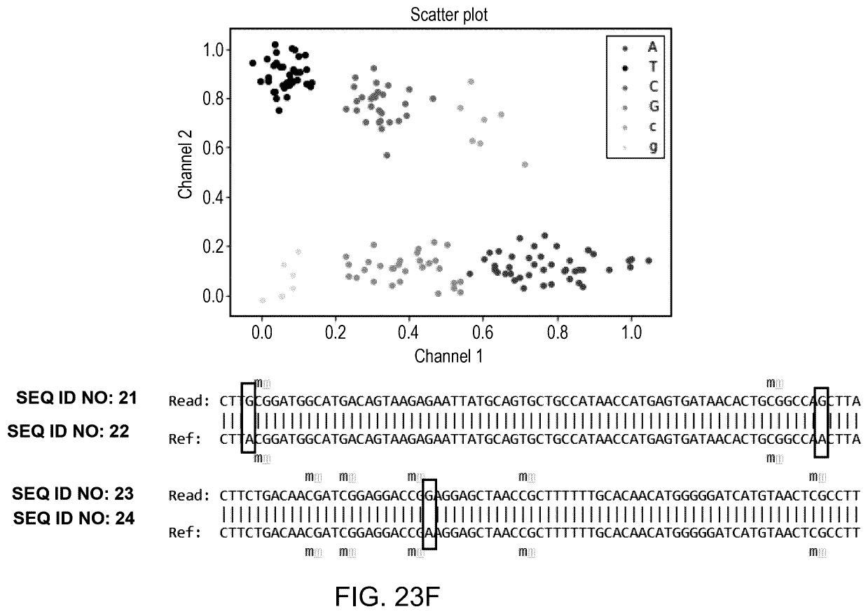

The 9 QaM results are shown in FIGS. 23A to 23F for six different library fragments, where modified cytosines can be identified by characteristic clouds in the top right corner and the bottom left corner in the plot. If the original strands in the library contained a (5mC)-G base pair (the first base corresponding to the forward strand of the library polynucleotide, and the second base corresponding to the reverse strand of the library polynucleotide), this corresponds to a C-G base pair after bisulfite conversion. As such, the forward strand of the template provides a C read (as the forward strand of the template has a G at the corresponding position), and the reverse complement strand of the template provides a C read too (as the reverse complement strand of the template has a G at the corresponding position too), which therefore appears in the top right corner of the plots in FIGS. 23A to 23F (a (C,C) read).

In addition, if the original strands in the library contained a G-(5mC) base pair (the first base corresponding to the forward strand of the library polynucleotide, and the second base corresponding to the reverse strand of the library polynucleotide), this corresponds to a G-C base pair after bisulfite conversion. As such, the forward strand of the template provides a G read (as the forward strand of the template has a C at the corresponding position), and the reverse complement strand of the template provides a G read too (as the reverse complement strand of the template has a C at the corresponding position too), which therefore appears in the bottom left corner of the plots in FIGS. 23A to 23F (a (G,G) read).

By contrast, if the original strands in the library contained a C-G base pair (the first base corresponding to the forward strand of the library polynucleotide, and the second base corresponding to the reverse strand of the library polynucleotide), this corresponds to a T-G mismatched base pair after bisulfite conversion (where C is converted to U, and U is read as T). As such, the forward strand of the template provides a T read (as the forward strand of the template has an A at the corresponding position), and the reverse complement strand of the template provides a C read (as the reverse complement strand of the template has a G at the corresponding position), which therefore appears in the top middle portion of the plots in FIGS. 23A to 23F (a (T,C) read).

If the original strands in the library contained a G-C base pair (the first base corresponding to the forward strand of the library polynucleotide, and the second base corresponding to the reverse strand of the library polynucleotide), this corresponds to a G-T mismatched base pair after bisulfite conversion (where C is converted to U, and U is read as T). As such, the forward strand of the template provides a G read (as the forward strand of the template has a C at the corresponding position), and the reverse complement strand of the template provides an A read (as the reverse complement strand of the template has a T at the corresponding position), which therefore appears in the bottom middle portion of the plots in FIGS. 23A to 23F (a (G,A) read).

If the original strands in the library contained a T-A base pair (the first base corresponding to the forward strand of the library polynucleotide, and the second base corresponding to the reverse strand of the library polynucleotide), this remains as a T-A base pair after bisulfite conversion. As such, the forward strand of the template provides a T read (as the forward strand of the template has an A at the corresponding position), and the reverse complement strand of the template provides a T read too (as the reverse complement strand of the template has an A at the corresponding position too), which therefore appears in the top left corner of the plots in FIGS. 23A to 23F (a (T,T) read).

Finally, if the original strands in the library contained an A-T base pair (the first base corresponding to the forward strand of the library polynucleotide, and the second base corresponding to the reverse strand of the library polynucleotide), this remains as an A-T base pair after bisulfite conversion. As such, the forward strand of the template provides an A read (as the forward strand of the template has a T at the corresponding position), and the reverse complement strand of the template provides an A read too (as the reverse complement strand of the template has a T at the corresponding position too), which therefore appears in the bottom right corner of the plots in FIGS. 23A to 23F (an (A,A) read).

| Library | Accuracy | Sensitivity | Specificity |
|---|---|---|---|
| Library fragment 1 | 85/85 (100%) | 10/10 (100%) | 75/75 (100%) |
| (FIG. 23A) | | | |
| Library fragment 2 | 72/72 (100%) | 10/10 (100%) | 62/62 (100%) |
| (FIG. 23B) | | | |
| Library fragment 3 | 73/73 (100%) | 10/10 (100%) | 63/63 (100%) |
| (FIG. 23C) | | | |
| Library fragment 4 | 148/150 (98.67%) | 17/18 (94.44%) | 133/133 (100%) |
| (FIG. 23D) | | | |
| Library fragment 5 | 148/150 (98.67%) | 14/14 (100%) | 136/136 (100%) |
| (FIG. 23E) | | | |
| Library fragment 6 | 147/150 (98%) | 14/14 (100%) | 136/136 (100%) |
| (FIG. 23F) | | | |

(Accuracy = number of correct base calls (GCAT, irrespective of methylation status)/total number of bases;
Sensitivity = number of true positive methylated base calls/total number of methylated bases;
Specificity = number of true negative methylated base calls/(number of true negative methylated base calls + number of false positive methylated base calls))

Overall, these results show that methylation analysis can be conducted on polynucleotide sequences to identify modified cytosines. In particular, by enabling concurrent sequencing of the forward and reverse complement strands of the template (or reverse and forward complement strands of the template), modified cytosines can be identified quickly and accurately. Again, such a process is made viable by using the methods of preparing polynucleotide libraries as described herein.

SEQUENCE LISTING

SEQ ID NO: 1: P5 sequence
AATGATACGGCGACCACCGAGATCTACAC
SEQ ID NO: 2: P7 sequence
CAAGCAGAAGACGGCATACGAGAT
SEQ ID NO: 3: P5′ sequence (complementary to P5)
GTGTAGATCTCGGTGGTCGCCGTATCATT
SEQ ID NO: 4: P7′ sequence (complementary to P7)
ATCTCGTATGCCGTCTTCTGCTTG
SEQ ID NO: 5: Alternative P5 sequence
AATGATACGGCGACCGA
SEQ ID NO: 6: Alternative P5′ sequence
(complementary to alternative P5 sequence)
TCGGTCGCCGTATCATT

1. A method of determining a polynucleotide sequence, the method comprising:
preparing a polynucleotide library comprising a double-stranded polynucleotide fragment and adaptor, the double-stranded polynucleotide fragment comprising a first strand having a first sequence of nucleotides and a second strand having a second sequence of nucleotides that is complementary to the first sequence of nucleotides;
amplifying the polynucleotide library to generate a plurality of template strands on a solid support, the plurality of template strands comprising a first portion that includes the first sequence of nucleotides and a second portion that includes a complement of the second sequence of nucleotides;
sequencing the first portion and the second portion of the plurality of template strands concurrently, the sequencing comprising:
contacting the plurality of template strands on the solid support with a plurality of labelled nucleotides, wherein a polymerase incorporates one or more types of labelled nucleotides complementary to at least the first portion or the second portion of the plurality of template strands;
acquiring intensity data comprising a combined intensity of signal emitted by the one or more types of labelled nucleotides in response to illumination by a light source; and
processing the intensity data to determine sequence information associated with the double-stranded polynucleotide fragment, wherein the intensity data is used to identify a match or a mismatch between the first portion of the plurality of template strands and the second portion of the plurality of template strands.

2. The method of claim 1, wherein the plurality of labelled nucleotides comprises different types of labelled nucleotides that are delivered to the solid support sequentially, and
wherein the intensity data is acquired after each delivery of a different type of labelled nucleotides.

3. The method of claim 1, wherein the plurality of labelled nucleotides comprises different types of labelled nucleotides that are delivered to the solid support simultaneously.

4. The method of claim 1, wherein the adapter comprises (a) two adapter strands having at least one base-paired region and a non-complementary region, or (b) two adapter strands having a first base-paired region and a second base-paired region, and
wherein the first base-paired region and the second base-paired region are separated by the non-complementary region.

5. The method of claim 1, wherein the combined intensity of signals is a combined intensity of co-localized optical signals.

6. The method of claim 5, wherein the co-localized optical signals are not spatially resolved on the solid support.

7. The method of claim 1, wherein the plurality of labelled nucleotides comprises different types of labelled nucleotides configured to emit optical signals for detection by an optical system having one optical channel, wherein the intensity data is acquired from optical signals detected in the one optical channel.

8. The method of claim 1, wherein the plurality of labelled nucleotides comprises different types of labelled nucleotides configured to emit optical signals for detection by an optical system having two optical channels.

9. The method of claim 8, wherein the different types of labelled nucleotides comprise a first type of labelled nucleotides and a second type of labelled nucleotides, and
wherein the first type of labelled nucleotides and the second type of labelled nucleotides each comprise a spectrally distinct label.

10. The method of claim 9, wherein the intensity data is acquired from optical signals detected in at least one of the two optical channels.

11. The method of claim 1, wherein the plurality of labelled nucleotides comprises different types of labelled nucleotides configured to emit optical signals for detection by an optical system having four optical channels, each different type of labelled nucleotides comprising a spectrally distinct label.

12. The method of claim 1, wherein the polymerase incorporates the same type of labelled nucleotides complementary to the first portion of the plurality of template stands and the second portion of the plurality of template strands, and
wherein the intensity data is used to identify a match between the first portion of the plurality of template strands and the second portion of the plurality of template strands.

13. The method of claim 12, wherein the intensity data is used to determine a base call.

14. The method of claim 1, wherein the polymerase does not incorporate the same type of labelled nucleotides complementary to the first portion of the plurality of template stands and the second portion of the plurality of template strands, and
wherein the intensity data is used to identify a mismatch between the first portion of the plurality of template strands and the second portion of the plurality of template strands.

15. The method of claim 14, wherein the intensity data is not used to determine a base call.

16. The method of claim 1, wherein the first portion and the second portion of the plurality of template strands are sequenced in the same direction.

17. The method of claim 1, wherein using the intensity data to identify a match comprises mapping the signals emitted by the one or more types of labelled nucleotides to a distribution of intensity data representing the match.

18. The method of claim 1, wherein a match is identified when combined signal intensities from the first portion and the second portion indicate the same base is present at a position in the first portion and the second portion.

19. The method of claim 1, wherein using the intensity data to identify a mismatch comprises mapping the signals emitted by the one or more types of labelled nucleotides to a distribution of intensity data representing the mismatch.

20. The method of claim 19, wherein a mismatch is identified when combined signal intensities from the first portion and the second portion indicate the base present in the first portion is different from the base present at that position in the second portion.

21. A method of determining a polynucleotide sequence, the method comprising:
providing an amplified polynucleotide library comprising a plurality of template strands on a solid support;
hybridizing sequencing primers to the plurality of template strands on the solid support, wherein the sequencing primers hybridize to a first strand and the reverse complement of a second strand to sequence in the same direction on the template strands;
sequencing the plurality of template strands on the solid support using a polymerase to incorporate a plurality of labelled nucleotides comprising one or more types of labelled nucleotides;
acquiring intensity data comprising a combined intensity of the signals emitted by the one or more types of labelled nucleotides incorporated; and
processing the intensity data to determine sequence information associated with the template strands, wherein library preparation errors and sequencing errors are removed from the sequence information.

22. The method of claim 21, wherein the plurality of labelled nucleotides comprises different types of labelled nucleotides that are delivered to the solid support sequentially, and
wherein the intensity data is acquired after each delivery of a different type of labelled nucleotides.

23. The method of claim 21, wherein the plurality of labelled nucleotides comprises different types of labelled nucleotides that are delivered to the solid support simultaneously.

24. The method of claim 21, wherein the intensity of the signals emitted from both the first strand and the complement of the second strand identifies a base call at a position in a DNA sequence, the base call identifying a variant nucleotide in the sequence.

25. The method of claim 21, wherein the intensity data is used to identify a mismatch between the first strand and the complement of the second strand, wherein the mismatch is an error introduced during library preparation or sequencing.

26. The method of claim 21, wherein the plurality of labelled nucleotides comprises different types of labelled nucleotides configured to emit optical signals for detection by an optical system having one optical channel.

27. The method of claim 21 wherein the different types of labelled nucleotides comprise a first type of labelled nucleotides and a second type of labelled nucleotides, and
wherein the first type of labelled nucleotides and the second type of labelled nucleotides each comprise a spectrally distinct label.

28. The method of claim 21, wherein the same type of labelled nucleotides are incorporated into the complement to the first strand and the copy of the second strand within the plurality of template strands, and
wherein the intensity data is used to identify a match between the first strands and the copy of the second strands of the plurality of template strands.

29. The method of claim 21, wherein the plurality of template strands are produced by ligating adaptors to a first and second strand of a target polynucleotide and performing polymerase chain reaction to produce the amplified polynucleotide library comprising a plurality of template strands.

30. A method of determining a polynucleotide sequence, the method comprising:
providing an amplified polynucleotide library comprising a plurality of template strands on a solid support, wherein the solid support is a bead, a well, or a flow cell surface;
hybridizing sequencing primers to the plurality of template strands on the solid support, wherein the sequencing primers hybridize to a first strand and the reverse complement of a second strand to sequence in the same direction on the template strands;
sequencing the plurality of template strands on the solid support by adding a plurality of labelled nucleotides comprising one or more types of labelled nucleotides in the presence of a polymerase to incorporate the one or more labelled nucleotides into complementary nucleotide sequences of the plurality of template strands, wherein the plurality of labelled nucleotides comprises different types of labelled nucleotides configured to emit optical signals;
acquiring by an optical system intensity data comprising a combined intensity of the signals emitted by the one or more types of incorporated labelled nucleotides; and
processing the intensity data obtained to determine sequence information associated with the template strands,
wherein the intensity of the signals emitted from both the first strand and the complement of the second strand identifies a base call at a position in a DNA sequence, the base call identifying a variant nucleotide in the sequence, and
wherein library preparation errors and sequencing errors are removed from the sequence information.


SEQUENCE LISTING (24 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 24 bp, synthetic construct
  source 1..24: mol_type = other DNA
caagcagaagacggcatacgagat

SEQ ID NO: 3 - DNA, 29 bp, synthetic construct
  source 1..29: mol_type = other DNA
gtgtagatctcggtggtcgccgtatcatt

SEQ ID NO: 4 - DNA, 24 bp, synthetic construct
  source 1..24: mol_type = other DNA
atctcgtatgccgtcttctgcttg

SEQ ID NO: 5 - DNA, 17 bp, synthetic construct
  source 1..17: mol_type = other DNA
aatgatacggcgaccga

SEQ ID NO: 6 - DNA, 17 bp, synthetic construct
  source 1..17: mol_type = other DNA
tcggtcgccgtatcatt

SEQ ID NO: 7 - DNA, 70 bp, synthetic construct
  source 1..70: mol_type = other DNA
  misc_difference 69^70: note = phosphorothioate linkage
  modified_base 32: mod_base = OTHER; note = uracil
gctgaggatctcgtatgccgtcttctgcttgtaatgatacggcgaccaccgagatctacactcctcagct

SEQ ID NO: 8 - DNA, 69 bp, synthetic construct
  source 1..69: mol_type = other DNA
  modified_base 35: mod_base = OTHER; note = biotin-T
  misc_difference 68^69: note = phosphorothioate linkage
gaagagcacacgtctgaactccagtcactagggataacagggtaatctttccctacacgacgctcttct

SEQ ID NO: 9 - DNA, 70 bp, synthetic construct; identical to SEQ ID NO: 7
  source 1..70: mol_type = other DNA
  misc_difference 69^70: note = phosphorothioate linkage
  modified_base 2: mod_base = OTHER; note = 5-methylcytosine
  modified_base 10: mod_base = OTHER; note = 5-methylcytosine
  modified_base 12: mod_base = OTHER; note = 5-methylcytosine
  modified_base 18..19: mod_base = OTHER; note = 5-methylcytosine
  modified_base 22: mod_base = OTHER; note = 5-methylcytosine
  modified_base 25: mod_base = OTHER; note = 5-methylcytosine
  modified_base 28: mod_base = OTHER; note = 5-methylcytosine
  modified_base 32: mod_base = OTHER; note = uracil
  modified_base 40: mod_base = OTHER; note = 5-methylcytosine
  modified_base 43: mod_base = OTHER; note = 5-methylcytosine
  modified_base 46..47: mod_base = OTHER; note = 5-methylcytosine
  modified_base 49..50: mod_base = OTHER; note = 5-methylcytosine
  modified_base 56: mod_base = OTHER; note = 5-methylcytosine
  modified_base 59: mod_base = OTHER; note = 5-methylcytosine
  modified_base 61: mod_base = OTHER; note = 5-methylcytosine
  modified_base 63..64: mod_base = OTHER; note = 5-methylcytosine
  modified_base 66: mod_base = OTHER; note = 5-methylcytosine
  modified_base 69: mod_base = OTHER; note = 5-methylcytosine

SEQ ID NO: 10 - DNA, 69 bp, synthetic construct; identical to SEQ ID NO: 8
  source 1..69: mol_type = other DNA
  misc_difference 68^69: note = phosphorothioate linkage
  modified_base 9: mod_base = OTHER; mod_base = OTHER; note = 5-methylcytosine
  modified_base 11: mod_base = OTHER; mod_base = OTHER; note = 5-methylcytosine
  modified_base 14: mod_base = OTHER; mod_base = OTHER; note = 5-methylcytosine
  modified_base 19: mod_base = OTHER; mod_base = OTHER; note = 5-methylcytosine
  modified_base 21: mod_base = OTHER; mod_base = OTHER; note = 5-methylcytosine
  modified_base 22: mod_base = OTHER; mod_base = OTHER; note = 5-methylcytosine
  modified_base 26: mod_base = OTHER; mod_base = OTHER; note = 5-methylcytosine
  modified_base 28: mod_base = OTHER; mod_base = OTHER; note = 5-methylcytosine
  modified_base 35: mod_base = OTHER; mod_base = OTHER; note = Biotin-T
  modified_base 38: mod_base = OTHER; mod_base = OTHER; note = 5-methylcytosine
  modified_base 47: mod_base = OTHER; mod_base = OTHER; note = 5-methylcytosine
  modified_base 51: mod_base = OTHER; mod_base = OTHER; note = 5-methylcytosine
  modified_base 52: mod_base = OTHER; mod_base = OTHER; note = 5-methylcytosine
  modified_base 53: mod_base = OTHER; mod_base = OTHER; note = 5-methylcytosine
  modified_base 56: mod_base = OTHER; mod_base = OTHER; note = 5-methylcytosine
  modified_base 58: mod_base = OTHER; note = 5-methylcytosine
  modified_base 61: mod_base = OTHER; mod_base = OTHER; note = 5-methylcytosine
  modified_base 63: mod_base = OTHER; mod_base = OTHER; note = 5-methylcytosine
  modified_base 65: mod_base = OTHER; note = 5-methylcytosine
  modified_base 68: mod_base = OTHER; mod_base = OTHER; note = 5-methylcytosine
  modified_base 7: mod_base = OTHER; note = 5-methylcytosine

SEQ ID NO: 11 - DNA, 85 bp, synthetic construct
  source 1..85: mol_type = other DNA
  modified_base 11: mod_base = OTHER; note = 5-Methylcytosine
  modified_base 17: mod_base = OTHER; note = 5-Methylcytosine
  modified_base 24: mod_base = OTHER; note = 5-Methylcytosine
  modified_base 41: mod_base = OTHER; note = 5-Methylcytosine
  modified_base 65: mod_base = OTHER; note = 5-Methylcytosine
ccagtgaattcgagctcggtacccggggatcctctagagtcgacctgcaggcatgcaagcttggcgtaatcatggtcatagctgt

SEQ ID NO: 12 - DNA, 72 bp, synthetic construct
  source 1..72: mol_type = other DNA
  modified_base 21: mod_base = OTHER; note = 5-Methylcytosine
  modified_base 28: mod_base = OTHER; note = 5-Methylcytosine
  modified_base 46: mod_base = OTHER; note = 5-Methylcytosine
  modified_base 49: mod_base = OTHER; note = 5-Methylcytosine
  modified_base 65: mod_base = OTHER; note = 5-Methylcytosine
tggtatctttatagtcctgtcgggtttcgccacctctgacttgagcgtcgatttttgtgatgctcgtcaggg

SEQ ID NO: 13 - DNA, 73 bp, synthetic construct
  source 1..73: mol_type = other DNA
  modified_base 21: mod_base = OTHER; note = 5-Methylcytosine
  modified_base 28: mod_base = OTHER; note = 5-Methylcytosine
  modified_base 46: mod_base = OTHER; note = 5-Methylcytosine
  modified_base 49: mod_base = OTHER; note = 5-Methylcytosine
  modified_base 65: mod_base = OTHER; note = 5-Methylcytosine
tggtatctttatagtcctgtcgggtttcgccacctctgacttgagcgtcgatttttgtgatgctcgtcagggg

SEQ ID NO: 14 - DNA, 75 bp, synthetic construct
  source 1..75: mol_type = other DNA
  modified_base 17: mod_base = OTHER; note = 5-Methylcytosine
  modified_base 19: mod_base = OTHER; note = 5-Methylcytosine
  modified_base 30: mod_base = OTHER; note = 5-Methylcytosine
  modified_base 38: mod_base = OTHER; note = 5-Methylcytosine
  modified_base 41: mod_base = OTHER; note = 5-Methylcytosine
  modified_base 56: mod_base = OTHER; note = 5-methylcytosine
  modified_base 60: mod_base = OTHER; note = 5-methylcytosine
  modified_base 63: mod_base = OTHER; note = 5-Methylcytosine
aagttctgctatgtggcgcggtattatcccgtattgacgccgggcaagagcaactcggtcgccgcatacactatt

SEQ ID NO: 15 - DNA, 75 bp, synthetic construct
  source 1..75: mol_type = other DNA
ctcagaatgacttggttgagtactcaccagtcacagaaaggcatcttacggatggcatgacagtaagagaattat

SEQ ID NO: 16 - DNA, 75 bp, synthetic construct
  source 1..75: mol_type = other DNA
  modified_base 49: mod_base = OTHER; note = 5-Methylcytosine
ctcagaatgacttggttgagtactcaccagtcacagaaaagcatcttacggatggcatgacagtaagagaattat

SEQ ID NO: 17 - DNA, 75 bp, synthetic construct
  source 1..75: mol_type = other DNA
  modified_base 56: mod_base = OTHER; note = 5-Methylcytosine
ggcatgacagtaagagaattatgcagtgctgccataaccatgagtgataacactgcggccaacttacttctggca

SEQ ID NO: 18 - DNA, 75 bp, synthetic construct
  source 1..75: mol_type = other DNA
  modified_base 56: mod_base = OTHER; note = 5-Methylcytosine
ggcatgacagtaagagaattatgcagtgctgccataaccatgagtgataacactgcggccaacttacttctgaca

SEQ ID NO: 19 - DNA, 75 bp, synthetic construct
  source 1..75: mol_type = other DNA
  modified_base 2: mod_base = OTHER; note = 5-Methylcytosine
  modified_base 6: mod_base = OTHER; note = 5-Methylcytosine
  modified_base 14: mod_base = OTHER; note = 5-Methylcytosine
  modified_base 61: mod_base = OTHER; note = 5-methylcytosine
  modified_base 70: mod_base = OTHER; note = 5-methylcytosine
acgatcggaggacccgaggagctaaccgcttttttgcacaacatgggggatcatgtaactcgccttgatcgttgg

SEQ ID NO: 20 - DNA, 75 bp, synthetic construct
  source 1..75: mol_type = other DNA
  modified_base 2: mod_base = OTHER; note = 5-methylcytosine
  modified_base 6: mod_base = OTHER; note = 5-Methylcytosine
  modified_base 14: mod_base = OTHER; note = 5-methylcytosine
  modified_base 27: mod_base = OTHER; note = 5-methylcytosine
  modified_base 61: mod_base = OTHER; note = 5-methylcytosine
  modified_base 70: mod_base = OTHER; note = 5-methylcytosine
acgatcggaggacccaaggagctaaccgcttttttgcacaacatgggggatcatgtaactcgccttgatcgttgg

SEQ ID NO: 21 - DNA, 75 bp, synthetic construct
  source 1..75: mol_type = other DNA
  modified_base 5: mod_base = OTHER; note = 5-methylcytosine
  modified_base 65: mod_base = OTHER; note = 5-Methylcytosine
cttgcggatggcatgacagtaagagaattatgcagtgctgccataaccatgagtgataacactgcggccagctta

SEQ ID NO: 22 - DNA, 75 bp, synthetic construct
  source 1..75: mol_type = other DNA
  modified_base 5: mod_base = OTHER; note = 5-methylcytosine
  modified_base 65: mod_base = OTHER; note = 5-methylcytosine
cttacggatggcatgacagtaagagaattatgcagtgctgccataaccatgagtgataacactgcggccaactta

SEQ ID NO: 23 - DNA, 74 bp, synthetic construct
  source 1..74: mol_type = other DNA
  modified_base 10: mod_base = OTHER; note = 5-Methylcytosine
  modified_base 14: mod_base = OTHER; note = 5-Methylcytosine
  modified_base 22: mod_base = OTHER; note = 5-Methylcytosine
  modified_base 35: mod_base = OTHER; note = 5-Methylcytosine
  modified_base 69: mod_base = OTHER; note = 5-Methylcytosine
ttctgacaacgatcggaggacccgaggagctaaccgcttttttgcacaacatgggggatcatgtaactcgcctt

SEQ ID NO: 24 - DNA, 74 bp, synthetic construct
  source 1..74: mol_type = other DNA
  modified_base 10: mod_base = OTHER; note = 5-Methylcytosine
  modified_base 14: mod_base = OTHER; note = 5-Methylcytosine
  modified_base 22: mod_base = OTHER; note = 5-Methylcytosine
  modified_base 35: mod_base = OTHER; note = 5-Methylcytosine
  modified_base 69: mod_base = OTHER; note = 5-Methylcytosine
ttctgacaacgatcggaggacccaaggagctaaccgcttttttgcacaacatgggggatcatgtaactcgcctt